(19) United States
(12) Patent Application Publication
(10) Pub. No.: US 2005/0032126 A1
(43) Pub. Date: Feb. 10, 2005

(54) Methods and apparatus for use in detection and quantitation of various cell types and use of optical bio-disc for performing same

(76) Inventors: James H. Coombs, Singapore (SG); John Francis Gordon, Irvine, CA (US); Brigitte C. Phan, Irvine, CA (US); Joseph Roby I. Urcia, Westminster, CA (US)

Correspondence Address: Donald Bollella;DB Technical Consulting, 126 Almador, Irvine, CA, 92614, US

(21) Appl. No.: 10/792,291

(22) Filed: Mar. 2, 2004

Related U.S. Application Data
(60) Provisional application No. 60/451,587, filed on Mar. 3, 2003.

(51) Int. Cl. G01N 33/53; G01N 33/567

(52) U.S. Cl. 435/7.2

(57) ABSTRACT

Methods and apparatus for conducting differential cell counts including leukocytes and use of optical bio-discs for performing such cell counts is disclosed. The bio-disc includes a substantially circular substrate having a center and an outer edge, an active layer associated with the substrate; a target zone disposed between the center and the outer edge; and a plurality of capture antibodies bound to the active layer such that the antibodies are immobilized on the active layer in the target zone.

### CROSS-REFERENCE TO RELATED APPLICATIONS

This application claims the benefit of priority from U.S. Provisional Application Ser. No. 60/451,587 filed Mar. 3, 2003 which is herein incorporated by reference in its entirety.

### BACKGROUND OF THE INVENTION

1. Field of the Invention

This invention relates in general to cellular assays and, in particular, to cellular assays conducted on optical bio-discs. More specifically, but without restriction to the particular embodiments hereinafter described in accordance with the best mode of practice, this invention relates to methods and apparatus for conducting differential cell counts including leukocytes and use of optical bio-discs for performing such cell counts.

2. Discussion of the Related Art

A number of research and diagnostic situations require isolation and analysis of specific cells from a mixture of cells. Particularly the source could be blood, spinal fluid, bone marrow, tumor homogenates, lymphoid tissue, and the like.

Blood cell counts are used during diagnosis, treatment, and follow-up to determine the health of the patient. Complete blood count (CBC) is a collection of tests including hemoglobin, hematocrit, mean corpuscular hemoglobin, mean corpuscular hemoglobin concentration, mean corpuscular volume, platelet count, and white blood cell count. Blood count is the enumeration of the red corpuscles and the leukocytes per cu. mm. of whole blood.

White Blood Cell Count (WBC, leukocytes) is the total number of white blood cells in a standard sample of blood. In a normal healthy person, typically the WBC counts are 4000 to 10800 cells per microliter (μL). Factors such as exercise, stress, and disease can affect these values. A high WBC may indicate infection, leukemia, or tissue damage. There is increased risk of infection if it falls below 1000 cells per microliter.

Leukocyte differential testing is essential to gather information beyond that obtainable from the leukocyte count itself. Leukocyte differential count is used to evaluate newly suspected infection or fever (even if the CBC is normal), suspicion of a disorder associated with abnormalities, an abnormal leukocyte count, suspected leukemia, other abnormalities such as eosinophilia, monocytosis and basophilia. Repeated testing for leukocyte or leukocyte differential may be performed in the presence of severe leukopenia (e.g., secondary to drug therapy). During treatment, for e.g. chemotherapy or radiation therapy, blood counts are very important to determine if the treatment is depleting healthy blood cells in addition to cancerous cells.

Differential leukocyte counts are determined by computerized cell counting equipment. The machine determines the total count and the percentages of the five major white cell types. In normal individuals, there are a majority of neutrophils (50-60%), followed by lymphocytes (2040%), then monocytes (2-9%), with a few eosinophils (14%) and basophils (0.5-2%).

Within the category of lymphocytes, there are further lymphocytes and further sub-types of cells. For example, lymphocytes can be broadly divided into T-cells (thymus-derived lymphocytes) and B-cells (bursal-equivalent lymphocytes), which are largely responsible for cell-mediated and humoral immunity respectively. Although morphological characteristics have been used to classify groups within the leukocytes, morphology alone has proved inadequate in distinguishing the many functional capabilities of lymphocyte sub-types. To distinguish lymphocytes with various functions, techniques including analysis by rosetting, immuno-fluorescence microscopy, enzyme histochemistry, and recently, monoclonal antibodies have been developed. T cells are distinguished by the presence of surface markers including two glycoproteins on their surface CD4 and CD8 (CD4+ T cells and CD8+T cells). CD4+ T helper cells are involved in antibody-mediated immunity. They bind to antigen presented by B cells. And the result is development of clone of plasma cells secreting antibodies against antigenic material. T cells are also essential for cell-mediated immunity. CD4+cells bind to antigen presented by antigen-presenting cells (APCs) like phagocytic macrophages and dendritic cells. The T cells then release lymphokines that attract other cells to the area. The result is inflammation, the accumulation of cells and molecules that attempt to wall off and destroy the antigenic material.

CD8+, cytotoxic/suppressor type cells secrete molecules that destroy the cell to which they have bound. This is a very useful function if the target cell is infected with a virus because the cell is usually destroyed before it can release a fresh crop of viruses which are able to infect other cells.

HIV and AIDS

Human immunodefiency virus a retrovirus has high affinity for CD4+ cells and therefore CD4 T cells are potent targets for the virus. Acquired immune deficiency syndrome (AIDS) provides a vivid and tragic illustration of the importance of CD4+ cells in immunity. The human immunodeficiency virus (HIV) binds to CD4 molecules and thus invades and infects CD4+ cells. As the disease progresses, the number of CD4+ cells declines below its normal range of about 1000 per microliter (ul). One of the explanations may be the unceasing effort of the patient's CD8+T cells to destroy the infected CD4+cells.

When the number of CD4+ cells in blood drop below 400 per microliter, the ability of the patient to mount an immune response declines dramatically. Not only patient becomes hypersuscepticle to pathogens that invade the body, but also microorganisms, especially bacteria that normally inhabit our tissues without harming us. Eventually the patient dies of opportunistic infections like Candidiasis, Cytomegalovirus, Herpes simplex viruses, Pneumocystis carinii, pneumonia, Toxoplasmosis, Tuberculosis and others.

The estimation of CD4+ and CD8+ T-cell numbers and the ratio of CD4+/CD8+ T-cells is useful to assess the immune health of human patients with immune-compromised diseases. Individuals having AIDS, for example, shows the importance of CD4+ T-cells in immunity. As the disease progresses, the number of CD4+ T-cells declines below its normal range of about 1000 cells per μl. As the patient's CD8+T-cells destroy the infected CD4+ T-cells, uninfected CD4+cells may undergo apoptosis. Thus, the ratio of CD4+ to CD8+T-cells provides a diagnostic marker for the progression of the disease. The U.S. Public Health Service recommends that CD4+levels be monitored every 3-6 months in all infected persons (40 million tests are done every year in 600 testing laboratories in the United States).

In addition to CD4 and CD8, there are many other cell surface antigens (e.g., CD3, CD16, CD19, CD45, CD56) which can be used to identify sub-types of lymphocytes. The ability to detect these cell surface antigens by antibody techniques has added a new dimension to diagnostic pathology, and a variety of techniques are available for the study of immunophenotypes of hematolymphoid disorders (e.g., AIDS, leukemias, and lymphomas). Conventional microimmuno-assays such as radio-immunoassays (RIA), enzyme-immunoassay (EIA), fluorescence-immunoassay (FIA) use an isotope, an enzyme or a fluorescent substance to detect the presence or absence of corresponding analytes.

Leukemia Immunophenotyping

Surface markers in leukemia aid in identifying the tumor lineage for diagnostic and prognostic purposes. Comprehensive leukemia phenotyping begins with a review of the clinical history and morphology and a panel of markers are selected for each case. In most cases the lineage can be identified as T-cell, B-cell, or myeloid and a diagnosis, or differential diagnosis, can be made.

The aim of leukemia phenotyping is to identify the cell type of the neoplastic process. This phenotypic identification should outline the cell lineage and level of maturation, as an aid to the classification of the leukemia or lymphoma. Further, this phenotypic identification should assist in the determination as to whether the cell population is normal or abnormal and in the detection of a previously characterized population of cells in a sample for monitoring the disease remission, development or recurrence.

Leukemia immunophenotyping is performed on blood or bone marrow specimens, however, other body fluids or tissues may be examined. Leukocytes obtained using RBC lysis method or density gradient isolation such as ficoll hypaque can be used. Where possible, a total leukocyte count and differential should be performed before processing, and the cell concentration adjusted accordingly.

Lymphoma Immunophenotypinq

Surface markers in lymphoma aid in identifying the tumor lineage for diagnostic and prognostic purposes. Comprehensive leukemia/lymphoma phenotyping begins with a review of the clinical history and morphology and a panel of markers are selected for each case. In most cases the lineage can be identified as T cell, B cell, or myeloid and a diagnosis, or differential diagnosis, is made.

The aim of lymphoma phenotyping is to identify the cell type of the neoplastic process. This phenotypic identification should outline the cell lineage and level of maturation, as an aid to the classification of the lymphoma. Further, this phenotypic identification assists in the determination as to whether the cell population is normal or abnormal and in the detection of a previously characterized population of cells in a sample for monitoring the disease remission, development, or recurrence.

A complete blood count (including white blood cell count) is performed. Blood cell count is an important index of the response of the disease to treatment. These counts are also important to learn the effects of drug treatment or radiation therapy. The normal white cell counts are about 4000 to 11,000 per cubic millimeter in the blood. If the total WBC count is over 11,000 cells/mm3, it is referred to as leukocytosis a normal response to infections of the body. A blood count helps to determine if a drug is working. Traditionally, cell counts are performed by expensive electronic counters, like the FACS scanner, that require technical expertise to perform the test. The patterns of each cell type indicate if lymphoma is present and the type of lymphoma.

Monoclonal Antibody Panels

Many laboratories use multicolor immunofluorescence although in some cases single color immunofluorescence may be adequate. Antibodies routinely included are CD2, CD3, CD5, CD10, CD11c, CD14, CD19, CD20, CD22, CD23, CD25, CD45, CD103, FMC7, Heavy chains, Kappa, and Lambda. If clinical or morphologic features suggest a “T” or “NK” lymphocyte disorder, then the following additional antibody combinations are also performed: CD3/CD4/CD8, CD7/CD5/HLA-DR, CD25/CD2/CD3, CD16/CD56/CD19, CD57/CD8/CD3, TCR alpha-beta/delta-gamma/CD3.

The ability to detect cell-associated antigens by antibody techniques has added a new dimension to diagnostic pathology. A variety of techniques are available for the study of immunophenotypes of hematolymphiod disorders. However, further development of immunoassay methods utilizing an antibody-antigen reaction in a global detection method for a number of diseases including virus based disease such as acquired immune deficiency syndrome and T-Cell leukemia as well as various cancers, need to be developed. As would be apparent to one of skill in the art, the assay methods and optical bio-disc systems of the current invention may be used to perform such immunoassays.

Conventional microimmunoassays like radio-immunoassays (RIA), enzyme-immunoassay (EIA), fluorescence-immunoassay (FIA) use an isotope, an enzyme or a fluorescent substance in order to detect the presence or absence of corresponding antibodies or antigens, respectively, that react specifically therewith. However the above methods have limitations and disadvantages. RIA requires special installations, precautions, limited half-life and various other factors. Methods using enzyme or fluorescence substances as labels is measured by determining coloring or luminescence require sensitive, sophisticated instruments to detect the calorimetric or fluorescent reactions in addition to requiring several washing steps to remove excess, unbound, un-reacted reagents. Furthermore, application of the above methods of detection for cells particularly lymphocytes and cancer cells and the like specimens, needs improvement in technology for the preparation, detection and analysis in high efficiency.

A powerful tool developed around the use of fluorescent antibody specific for cell-surface antigens is the technique of fluorescence-activated cell sorting (FACS). This is a very reliable, fast and sensitive method. Flow cytometer is the most practical method that is automated and quantitative. The foremost requirement of a sample for flow cytometric analysis is that the sample is in a monodisperse suspension and labeling desired cells with fluorescent markers. However, it is very high-priced test and the whole system requires handling by a trained technician in a clinical analysis laboratory and an expensive instrument. Monoclonal antibodies are used as discrete probes and flow cytometry for objective quantification of large number of cells.

In addition the fundamental disadvantage is that the cells once analyzed are no longer available for repeated analysis or additional investigation for example microscopic examinations of rare event cells. A number of alternative technologies have been developed that have advantages and disadvantages over flow cytometer and all introduce their own specific problem.

Surface marker analysis is an important laboratory tool, which has been particularly very useful in studying leukemias, lymphomas and immunodeficiency diseases. Antibody-based micro array technologies certainly are the state-of-the art technique, particularly in clinical diagnostics, for identification of specific antigens in the samples including blood and tissue samples. Most diagnostic tests require determination of only a limited panel of analytes (such as in of cancers, leukemia, lymphoma, thyroid disease, etc.). Therefore, the requirements by a miniaturized technology for only a very small amount of blood sample and the savings in time and cost of laboratory personnel, upon simultaneous measurement of all the clinically relevant parameters in a single test are likely to prove compelling attractive to hospital laboratories and point-of-care facilities due to its cost-effectiveness, labor effective and its simplicity.

As an alternative to prior art systems and methods for cell counting, we have developed a simple, inexpensive system for analyzing, detecting, and quantitating cells, in particular blood cells, inclusive of the parasites and pathogens that infest the blood and other biological fluids like CSF. Related information and signal processing methods and software have been developed to identify various blood cells, parasites and pathogens.

As compared to prior methods and systems, we have developed a simple, miniaturized, ultra-sensitive, inexpensive system for cellular analysis. This system uses optical bio-discs, related detection assemblies, as well as information and signal processing methods and software.

### SUMMARY OF THE INVENTION

Micro technologies are very valuable particularly in clinical diagnostics for identification of cell types, parasites, pathogens and other biological matter. The present invention utilizes micro technologies to perform differential white cell counts in whole blood on optical bio-discs. In addition, this invention is directed to imaging blood cells, performing a differential white cell counts, and related processing methods and software.

The present test or assay can be performed in two ways. The first method is based upon the principle of optical imaging of blood cells in special channels located on the optical bio-disc. Approximately seven microliters of whole blood is injected into specially designed channels on the disc. The images are analyzed with cell recognition software that identifies these various leukocyte sub-types and generates a white cell differential count. The second method is based on specific cell capture using cell specific antibodies against specific cell, in this particular case antibodies directed against lymphocytes (CD4, CD2, CD19), monocytes (CD14), eosinophils (CD15) and so on. These leukocyte sub-type specific antibodies are assembled/attached to the solid surface within a bio-disc that includes a flow chamber.

To increase the specificity of cell type identification and quantitation, the captured cells may be tagged with microparticles or beads coated with specific antibodies directed to either the cell type of interest, or unwanted or contaminant cells to thereby from a bead-cell complex. This method allows for the differentiation between specific target cells and contaminant cells in the capture zone. Further details relating to the use of beads in identifying cells are discussed below in conjunction with FIGS. 18 to 24.

A bio-disc drive assembly is employed to rotate the disc, read and process any encoded information stored on the disc, and analyze the cell capture zones in the flow chamber of the bio-disc. The bio-disc drive is provided with a motor for rotating the bio-disc, a controller for controlling the rate of rotation of the disc, a processor for processing return signals from the disc, and analyzer for analyzing the processed signals. The rotation rate is variable and may be closely controlled both as to speed and time of rotation. The bio-disc may also be utilized to write information to the bio-disc either before or after the test material in the flow chamber and target zones is interrogated by the read beam of the drive and analyzed by the analyzer. The bio-disc may include encoded information for controlling the rotation of the disc, providing processing information specific to the type of cellular immunoassay to be conducted and for displaying the results on a monitor associated with the bio-drive.

Differential cell count protocol in general and in particular differential white blood cell counting protocol is developed for CD, CD-R, DVD, or DVD-R formats, modified versions of these formats, and alternatives thereto. The read or interrogation beam of the drive detects the various cells and bead-cell complexes in the analysis sample and generates images that can be analyzed with differential cell counter software.

Microscopic methods or sophisticated cell counters are essential to perform these tedious and laborious cell-counting assays. The present method uses optical bio-discs and its assemblies. Optical images of the various cell types and bead-cell complexes free in the analysis chamber or those captured by specific antibody capture method are generated and analyzed by cell recognition software program that identifies the various cellular elements in the blood or other body fluids by their light scattering properties. The present method may not require any processing of the sample prior to analysis like cell staining, RBC elimination and other laborious protocols. These methods include microscopic analysis or cell detection using an optical disc reader with a top-detector, bottom-detector, event counter, or cell counter described below in detail in conjunction with the drawing figures.

To further increase the accuracy and precision of the differential cell counting method of the present invention, different cell populations may be tagged or labeled. These tags may include, for example, microspheres, fluorescent labeled antibodies, and enzyme conjugated antibodies. Further details relating to other aspects associated with tagging or labeling of samples and/or reporter molecules is disclosed in, for example, commonly assigned co-pending U.S. patent application Ser. No. 10/121,281 entitled “Multi-Parameter Assays Including Analysis Discs and Methods Relating Thereto” filed Apr. 11, 2002, which is incorporated herein by reference in its entirety.

This invention or different aspects thereof may be readily implemented in or adapted to many of the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 16, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed November 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; and U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002.

All of these applications are herein incorporated by reference in their entireties. They thus provide background and related disclosure as support hereof as if fully repeated herein.

### BRIEF DESCRIPTION OF THE DRAWING

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1 is a pictorial representation of a bio-disc system according to the present invention;

FIG. 2 is an exploded perspective view of a reflective bio-disc as utilized in conjunction with the present invention;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc as employed in conjunction with the present invention;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17 is a pictorial flow chart showing the isolation of white blood cells using a gradient cell separation method and the analysis of a blood sample using the methods of the present invention;

FIG. 18 is a pictorial illustration of labelling a cell with a bead;

FIG. 19 is a pictorial representation of an embodiment of the present invention depicting the use of beads to prevent binding of unwanted cells to capture agents on a bio-disc;

FIGS. 20A and 20B present a graphic depiction of another embodiment of the present invention illustrating steps of a method for identifying various types of cells in a sample using various beads to specifically tag or label target cells immobilized on the bio-disc;

FIG. 21 is a pictorial representation of the use of beads to capture a micro-organism of interest and detect its presence using the optical bio-disc;

FIG. 22 is an illustration of tagging unwanted cells using beads;

FIG. 23A is a graphical representation of a 1 micron reporter bead and a 5 micron cell linked together in a complex positioned relative to the tracks of an optical bio-disc according to the present invention;

FIG. 23B is a series of signature traces derived from the complex of FIG. 23A utilizing a detected signal from the optical drive according to the present invention;

FIG. 24 presents micrographs of unattached beads, unlabeled cells, and bead-cell complexes or labeled cells; and

FIGS. 25A and 25B are pictorial representations of another embodiment of the present invention showing steps of a method for differentiating unwanted cells from target cells using enzymes to identify unwanted cells.

### DESCRIPTION OF THE INVENTION

The present invention is directed to disc drive systems, optical bio-discs, and cell differentiation and quantitation assays. More specifically, but without restriction to the particular embodiments hereinafter described in accordance with the best mode of practice, this invention relates to methods for differentiation of various cell populations in a biological sample for cell quantitation including, for example, white blood cells and use of optical bio-discs for performing such cell quantitation. Each of these aspects of the present invention is discussed below in further detail.

Drive System and Related Discs

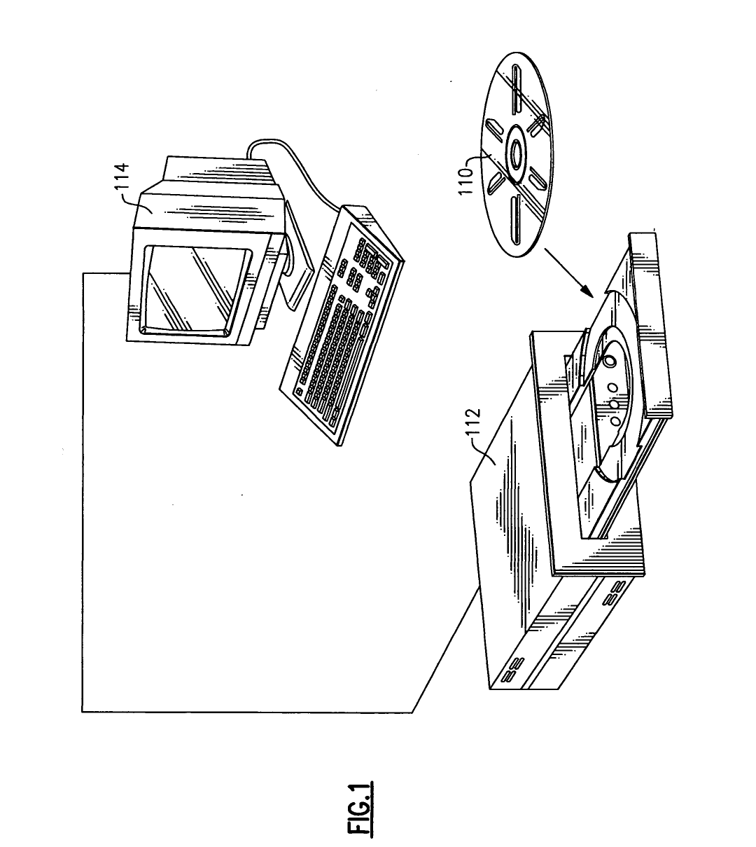

FIG. 1 is a perspective view of an optical bio-disc 110 according to the present invention as implemented to conduct the differential cell counts disclosed herein. The present optical bio-disc 110 is shown in conjunction with an optical disc drive 112 and a display monitor 114.

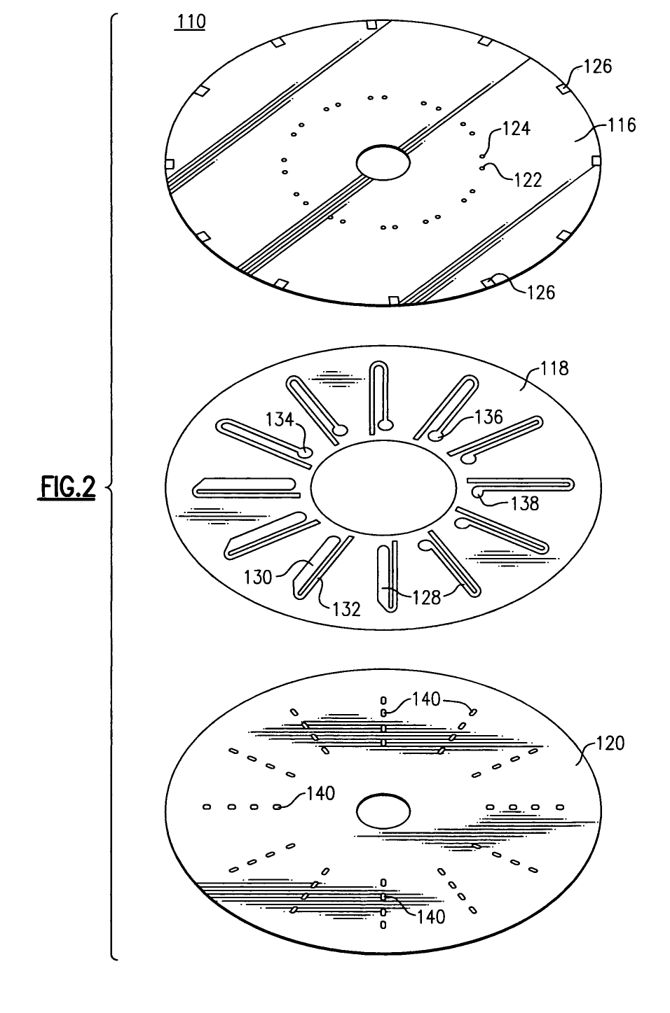

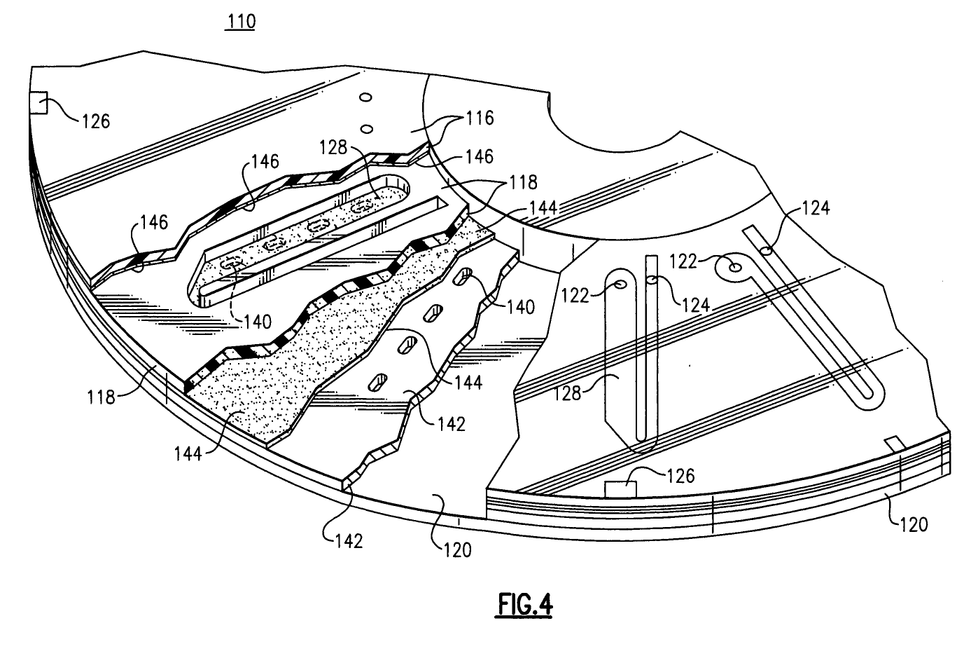

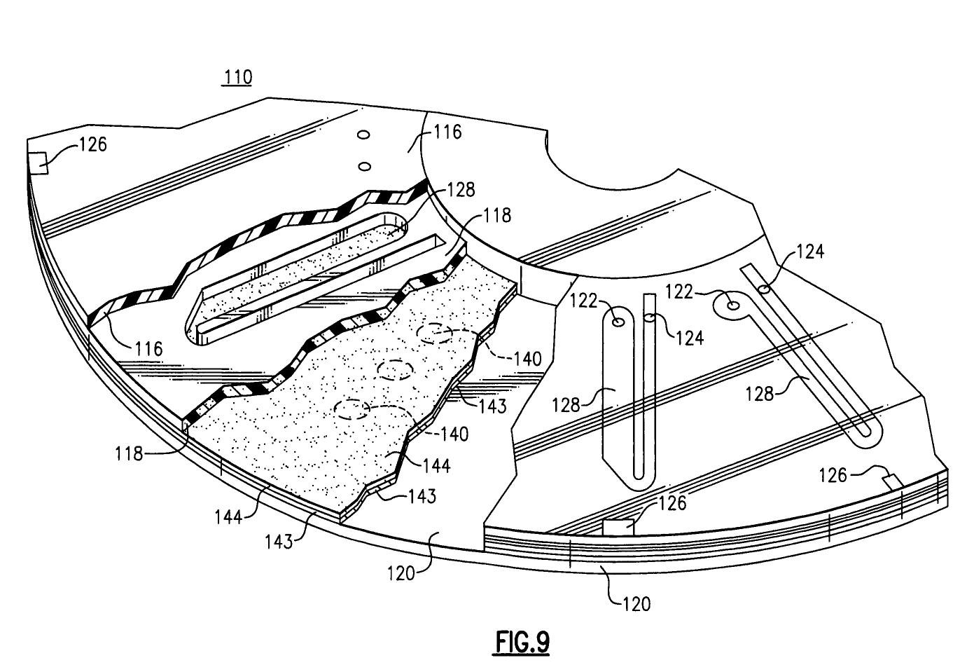

FIG. 2 is an exploded perspective view of the principle structural elements of one embodiment of the optical bio-disc 110. FIG. 2 is an example of a reflective zone optical bio-disc 110 (hereinafter “reflective disc”) that may be used in the present invention. The principle structural elements include a cap portion 116, an adhesive member 118, and a substrate 120. The cap portion 116 includes one or more inlet ports 122 and one or more vent ports 124. The cap portion 116 may be formed from polycarbonate and is preferably coated with a reflective surface 146 (as better illustrated in FIG. 4) on the bottom thereof as viewed from the perspective of FIG. 2. In the preferred embodiment, trigger markings 126 are included on the surface of the reflective layer 142 (as better illustrated in FIG. 4). Trigger markings 126 may include a clear window in all three layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10. The second element shown in FIG. 2 is an adhesive member 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated. The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

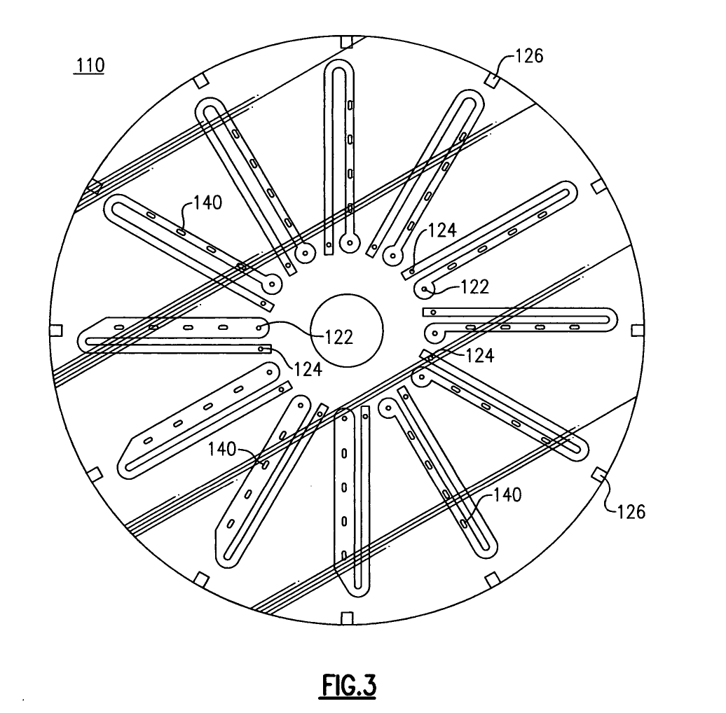

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the present invention. This view includes a portion of the various layers thereof, cut away to illustrate a partial sectional view of each principle, layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition hydrogels can be used. Alternatively other as illustrated in this embodiment, the plastic adhesive member 118 is applied over the active layer 144. The exposed section of the plastic adhesive member 118 illustrates the cut out or stamped U-shaped form that creates the fluidic circuits 128. The final principle structural layer in this reflective zone embodiment of the present bio-disc is the cap portion 116. The cap portion 116 includes the reflective surface 146 on the bottom thereof. The reflective surface 146 may be made from a metal such as aluminum or gold.

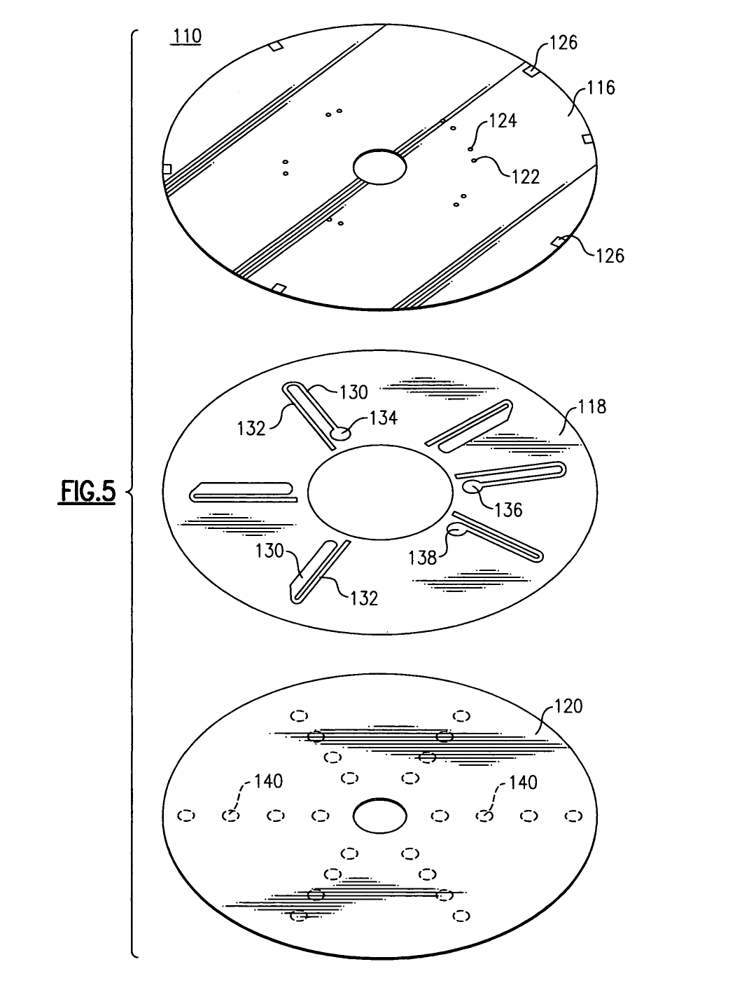

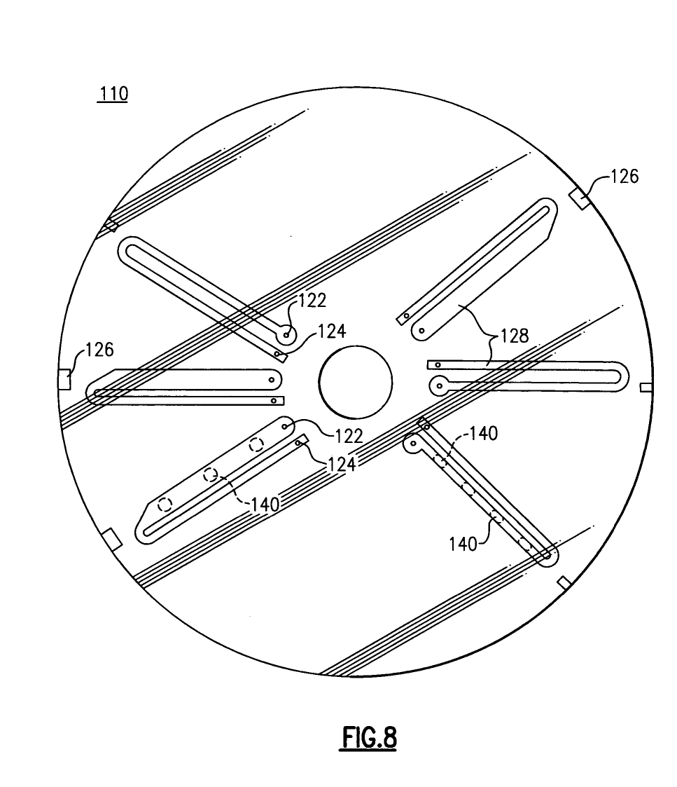

FIG. 5 is an exploded perspective view of the principal structural elements of a transmissive type of optical bio-disc 110 according to the present invention. The principle structural elements of the transmissive type of optical bio-disc 110 similarly include the cap portion 116, the adhesive member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

The third element illustrated in FIG. 5 is the substrate 120 which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIG. 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158, FIG. 10, while some of the light is reflected or returned back along the incident path. As indicated below, Table 1 presents the reflective and transmissive characteristics of a gold film relative to the thickness of the film. The gold film layer is fully reflective at a thickness greater than 800 Å. While the threshold density for transmission of light through the gold film is approximately 400 Å.

TABLE 1
Au film Reflection and Transmission (Absolute Values)

| Thickness | Thickness | | |
|---|---|---|---|
| (Angstroms) | (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

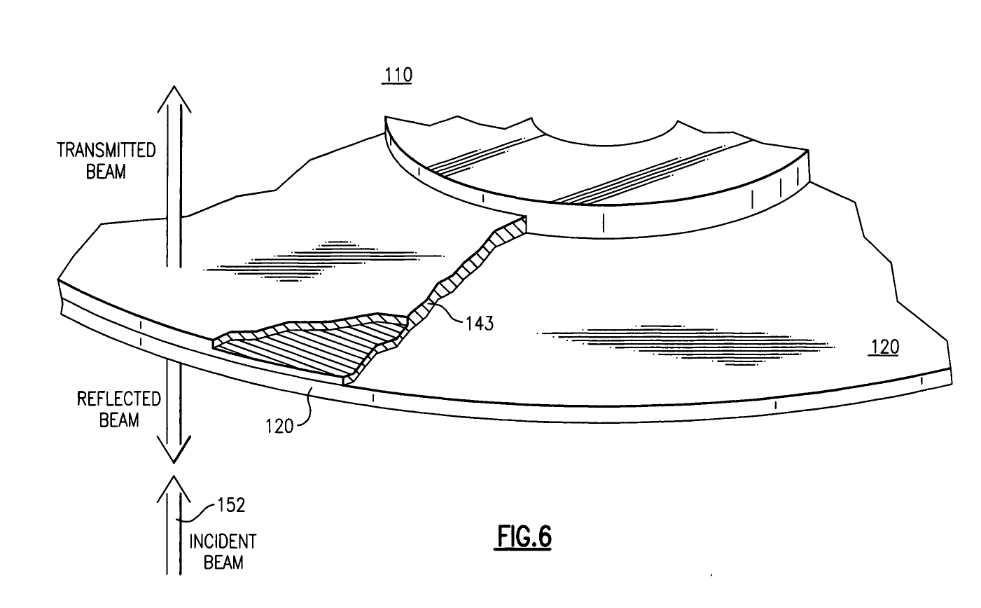

In addition to Table 1, FIG. 7 provides a graphical representation of the inverse proportion of the reflective and transmissive nature of the thin semi-reflective layer 143 based upon the thickness of the gold. Reflective and transmissive values used in the graph illustrated in FIG. 7 are absolute values.

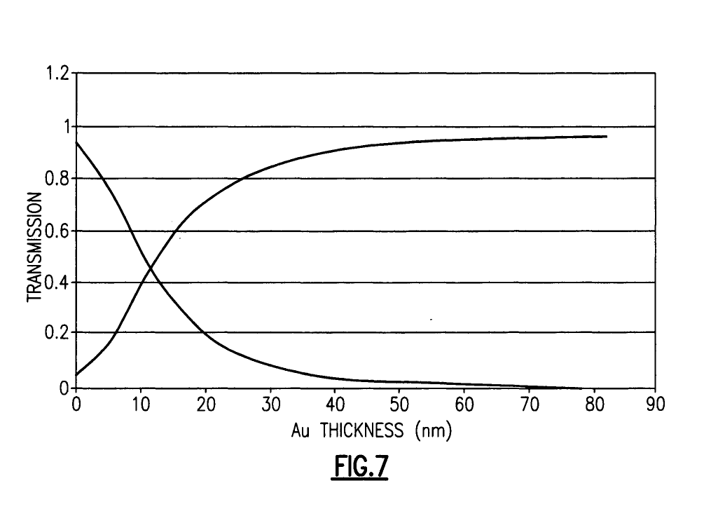

FIG. 8 is a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the present invention. The disc 110 is illustrated with a portion of the various layers thereof cut away to illustrate a partial sectional view of each principle, layer, substrate, coating, or membrane. FIG. 9 illustrates a transmissive disc format with the clear cap portion 116, the thin semi-reflective layer 143 on the substrate 120, and trigger markings 126. Trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 160, FIG. 10. FIG. 9 also shows, the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. An active layer 144 is applied over the thin semi-reflective layer 143. In the preferred embodiment, the active layer 144 is a 40 to 200 um thick layer of 2% polystyrene. Alternatively, polycarbonate, gold, activated glass, modified glass, or modified polystyrene, for example, polystyrene-co-maleic anhydride, may be used. In addition hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128. The final principle structural layer in this transmissive embodiment of the present bio-disc 110 is the clear, non-reflective cap portion 116 that includes inlet ports 122 and vent ports 124.

FIG. 10 is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal agents by a bottom detector 157. In the transmissive bio-disc format, on the other hand, the transmitted beam 156 is detected, by a top detector 158, and is also analyzed for the presence of signal agents. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 also illustrates a drive motor 162 and a controller 164 for controlling the rotation of the optical bio-disc 110. FIG. 10 further shows the processor 166 and analyzer 168 implemented in the alternative for processing the return beam 154 and transmitted beam 156 associated the transmissive optical bio-disc.

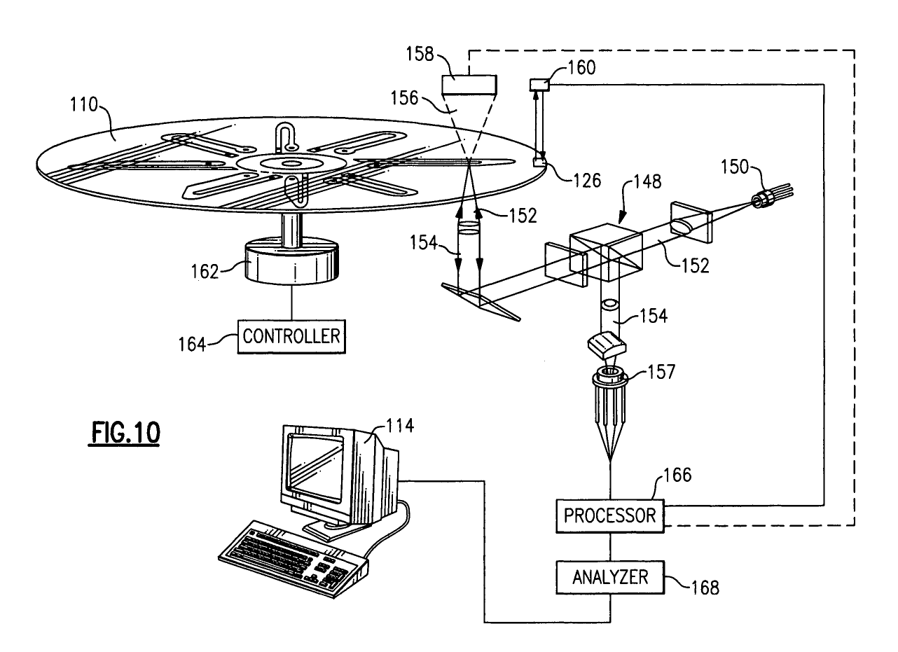

FIG. 11 is a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cut-out shapes, flow channel 130 is thereby formed. As indicated by the arrowheads shown in FIG. 1I, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

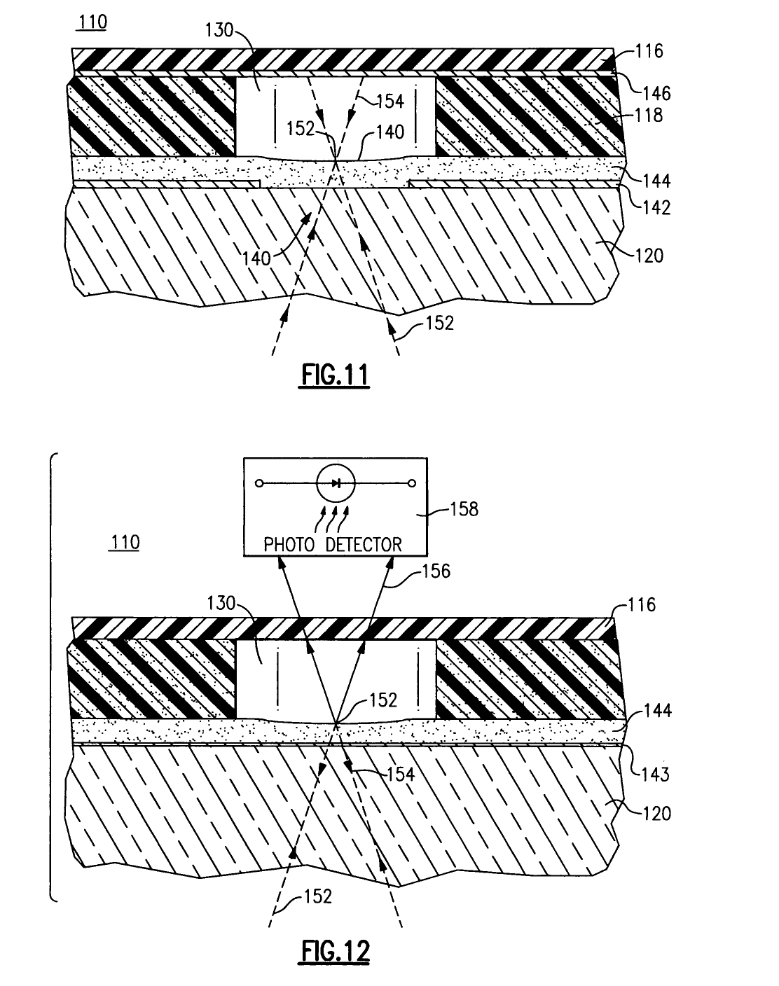

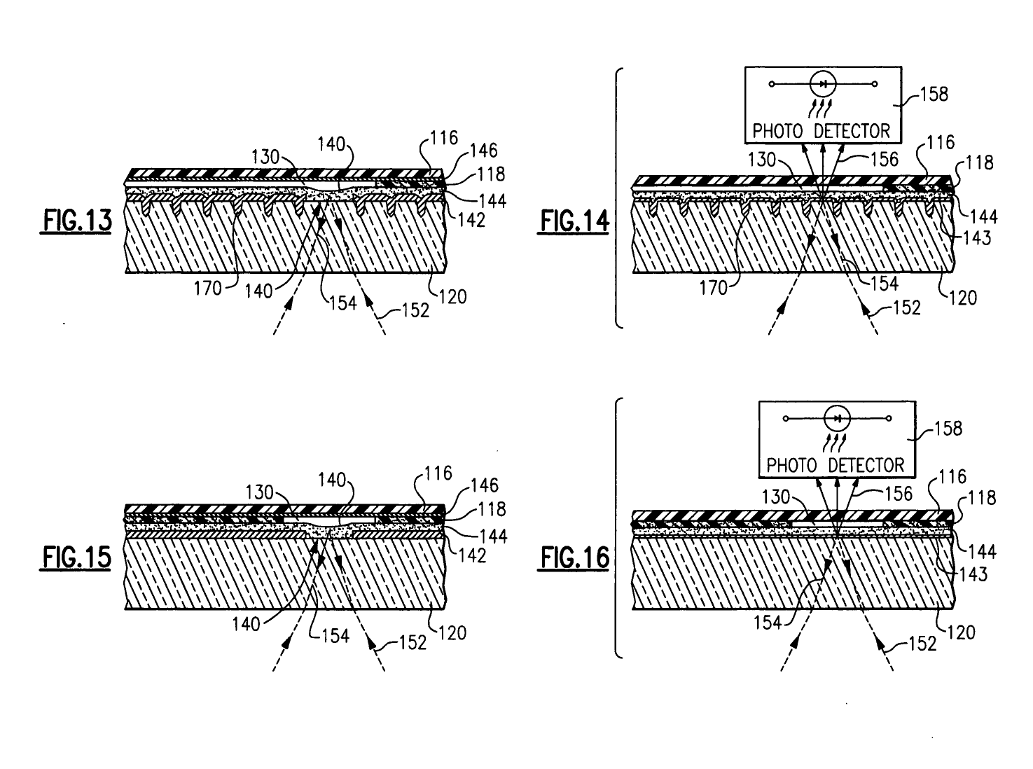

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a defined target zone 140 may or may not be present. Target zone 140 may be created by direct markings made on the thin semi-reflective layer 143 on the substrate 120. These marking may be done using silk screening or any equivalent method. In the alternative embodiment where no physical indicia are employed to define a target zone, the flow channel 130 in effect is utilized as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention, as described in FIG. 12. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

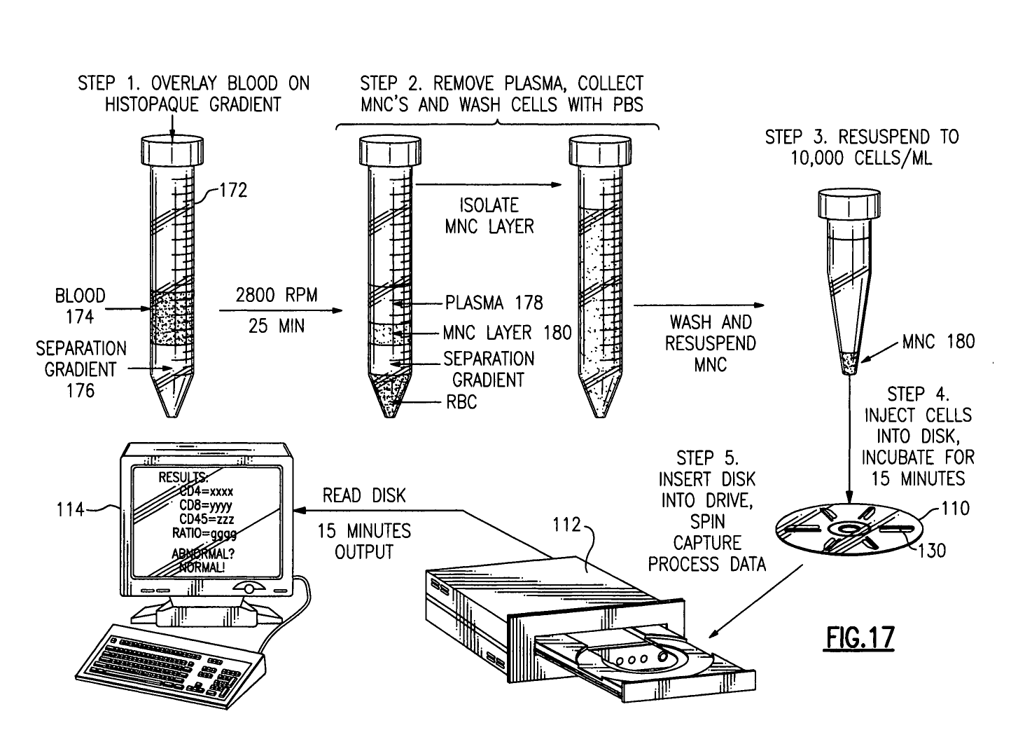

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

Isolating Cells of Interest from Whole Blood

FIG. 17 is a pictorial flow chart showing the preparation analysis of a blood sample for a cluster designation (CD) marker assay using the optical bio-disc system described above. In step 1, blood (4-8 ml) is collected directly into a 4 or 8 ml Becton Dickinson CPT Vacutainer™ and an anticoagulant such as EDTA, acid citrate dextrose (ACD), or heparin. In an equivalent step of another embodiment of the invention, 3 ml of blood in anticoagulant is overlayed into a tube 172 containing a separation gradient 176 such as Histopaque-1077 (Sigma Diagnostics, St. Louis, Mo.). In any case, the blood sample 174 is preferably used within two hours of collection. The tube 172 containing the separation gradient 176 with blood sample 174 overlay is centrifuged at 1500 to 1800 RCFs (2800 rpm) in a biohazard centrifuge with horizontal rotor and swing out buckets for 25 minutes at room temperature. After centrifugation, the plasma layer 178 is removed (step 2), leaving about 2 mm of plasma above the mononuclear cell (MNC) fraction 180. The MNC layer 180 is collected and washed with phosphate buffer saline (PBS). Cells are pelleted by centrifugation at 300 RCFs (1200 rpm) for 10 minutes at room temperature to remove any remaining platelets. The supernatant is removed and the MNC pellet 180 is re-suspended in PBS by tapping the tube gently. The final pellet 180 is re-suspended (step 3) and diluted with PBS to a cell count of 10,000-30,000 cells/ul depending upon the thickness of the flow channel 130 of the bio-disc 110. Alternatively, the T-lymphocytes may be isolated from whole blood using bio-active reagents that cause agglutination and precipitation of unwanted cells. A non-limiting example of a bio-active reagent is PrepaCyte (BioE, St. Paul, Minn.). PrepaCyte allows the isolation of T-lymphocytes from whole blood by selective removal of granulocytes, platelets, monocytes, B cells (up to 80%), natural killer (NK) cells (up to 80%). Further details relating to other aspects associated with the methods for isolating T-lymphocytes for use in an optical disc based cellular assay is disclosed in, for example, commonly assigned U.S. Provisional Patent Application Ser. No. 60/382,327 entitled “Methods for Isolation of Lymphocytes for Use in Differential Cell Counting and Use of Optical Bio-disc for Performing Same” filed May 22, 2002, which is incorporated herein by reference in its entirety.

Cellular Differentiation, Detection, and Quantitation

Cell capture by means of a surface coated with a single antibody results in (nearly) all cells containing that particular marker being captured. If the goal is to capture a specific cell type—for instance, T-Helper cells—then the use of a single marker may be insufficient. Techniques such as flow cytometry therefore use multiple markers, and only cells that contain all the chosen markers are counted. Therefore T-Helper and monocyte cells, both of which carry the CD4 antibody, are distinguished by means of a second marker such as CD3 (only CD4 cells) or CD19 (only monocytes). This gives a particular problem for surface capture methods with single antibodies, since it may be unavoidable that the monocytes are also captured.

Although it may be difficult to avoid secondary populations of cells from binding to the antibody coated surface, it is possible to distinguish them by marking them with a signal agent bound to a second antibody. For example, this signal agent may be a bead or a dye that absorbs light at a predetermined wavelength. The predetermined wavelength is preferably at or around the wavelength of the incident beam 152 of the optical disc reader 112. Therefore although both cell populations will be captured on the surface of the bio-disc, the cells will be distinguishable by the measurement system discussed above in conjunction with FIG. 10.

As an example, a CD4 antibody is deposited on the surface of the disc 110. It normally captures both a sub-population of CD4+ monocytes and CD4+ T-helper cells. However, during sample preparation, IR absorbent beads coated with a CD19 antibody are introduced, resulting in coating the monocytes with these beads since monocytes are CD19+while T-helper cells are not. This has two effects. Firstly, it reduces the binding probability of the monocytes to the CD4 capture area through steric hindrance. Secondly, the remaining monocytes that bind onto the capture area on the disc appear much darker to an IR laser beam than the CD4+ T-helper cells thereby allowing differentiation between monocytes and T-helper cells.

In particular, when the counting is being done by the bio-disc system of the present invention using hardware counting (based on S-curve recognition using a threshold value), the resultant monocyte absorption should be sufficient to reduce their S-curve amplitude below the threshold. Likewise, if the monocytes are distinguished with an IR dye, they appear darker and can be counted separately. Further details relating to hardware counting software and S-curve recognition are disclosed in commonly assigned, co-pending and related U.S. Provisional Patent Applications No. 60/356,982; 60/372,007 and 60/408,227; all entitled “Bio-Disc and Bio-Drive Analyser System Including Methods Relating Thereto” respectively filed on Feb. 13, 2002; Apr. 11, 2002; and Sep. 4, 2002. All of which are incorporated herein by reference in their entireties as if fully repeated herein.

These methods of enabling dual markers to be used-attaching an identifiable object or substance onto the surface of the cell, or coating the cell's surface to reduce its binding probability as discussed above-can be used with many types of cell assay systems. For instance, CD8 antibodies captures NK cells, which can be (non-uniquely, in this case) marked with CD56; or granulocytes can be marked specifically to distinguish them from lymphocytes on a CD45 antibody coated capture or target zone 140. Dyes need not be applied such that they only coat the relevant cells: they can also be absorbed non-specifically into the internal structure of the cell, such as the nucleus. Such cells could be used as calibrator elements on a capture zone if stabilized by fixation and pre-mixed in sample at a known concentration.

In principle, multiple markers can also be used. For instance, one signal agent may be a dye on a specific antibody, which binds to a marker on a cell. A second signal agent may be a micro-particle or bead on another specific antibody which binds to another marker. If the second marker is present, the beads will bind to the cell and prevent the cell from binding to the capture agents in the capture zone. If only the first, then the cells bind, but can be distinguished from the single-marker on the cells.

Positive identification is also possible. Cells with IR beads attached, or cells dyed using an IR dye, for example, which appear darker in comparison to other cells, when imaged using the optical disc reader of the present invention, can be counted directly. For instance, in a split-detector configuration used for real-time cell counting, the sum signal (A+B) rather than the difference signal (A-B) can be used to detect darker cells. Details relating to the split-detector used for real time cell counting and S-curve recognition are disclosed in commonly assigned and co-pending U.S. patent application Ser. No. 10/279,677 entitled “Segmented Area Detector for Biodrive and Methods Relating Thereto” filed on Oct. 24, 2002 which is herein incorporated by reference in its entirety. Further details relating to the use of multiple cell markers to identify one or more cells from a sample containing various cell types are discussed below.

Use of Micro-particles to Differentiate and Isolate Target Cells

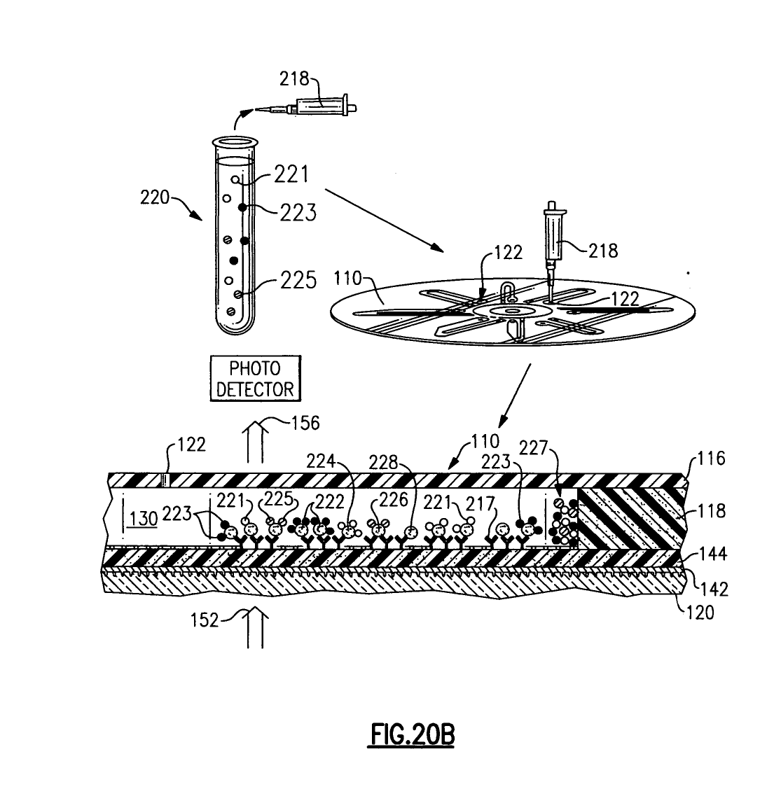

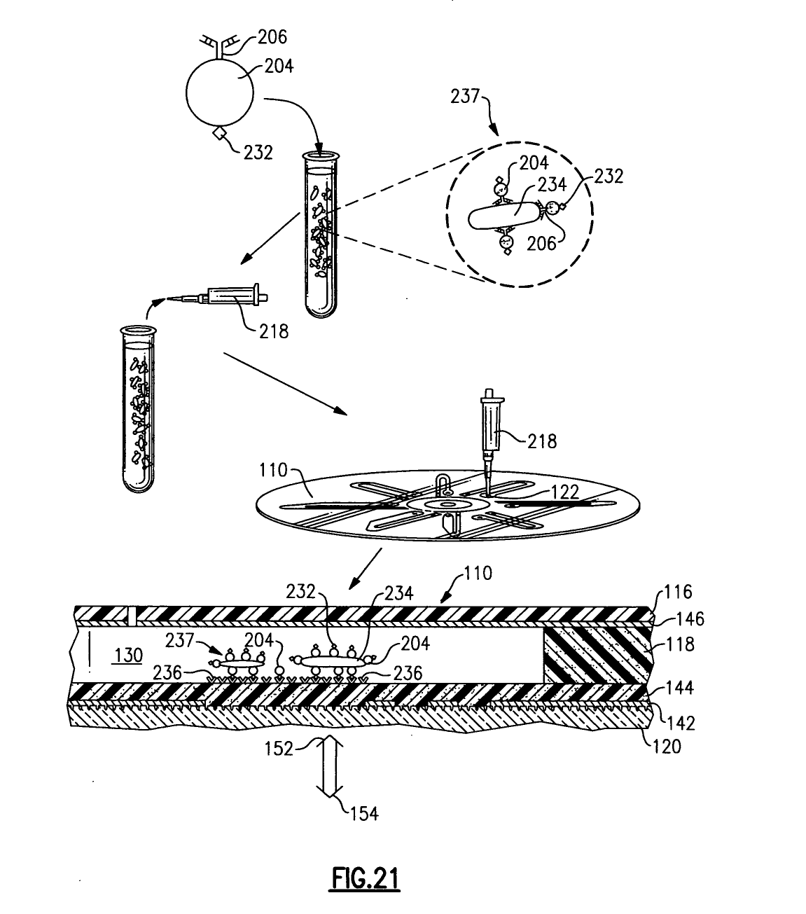

Lymphocyte subset immunophenotyping and quantitation through image analysis using the optical disc system described above, may require a secondary gate or parameter to increase the accuracy of cellular differentiation. The present invention relates to the use of reporter or signal agents such as beads or micro-particles of different physical properties, with or without a functionalyzed surface, conjugated with at least one signal antibody that specifically binds to target cells or unwanted cells to thereby form a bead-cell complex which is detectable by the optical disc reader. The signal antibody may be conjugated to the reporter or signal agent by a cross linker. Cross linkers include receptor-ligand interactions or binding agent-affinity agent interactions. The binding agent may be, for example, Streptavidin or Neutravidin. The affinity agent may be, for example, biotin. Alternatively, one of skill in the art would know that beads with a modified functionalyzed surface can be used to covalently conjugate the signal antibodies onto the bead surface and promote a rigid attachment of the bead or reporter agent to the cell of interest. It is also possible to use beads of different physical properties such as size, color, texture, reflectivity, absorbance, mass, fluorescence, phosphorescence, and/or magnetic properties to isolate and/or differentiate target cells. This process facilitates to better distinguish a target cell by a distinctive feature that is at least one type of reporter agent or bead attached to the cell surface as discussed below in conjunction with FIGS. 20A and 20B.

Inaccuracy results when two or more specific cell types share the same antigenic epitope on their membranes with other subsets or cell types such as T-cells and monocytes having CD4 antigens on their membranes, CD3 on all mature T cells, and CD45 on all leukocytes. This makes it difficult to differentiate cell types using a common capture antibody such as anti-CD4 or anti-CD45 without using tags or labels and the capability of measuring the cell morphology and size.

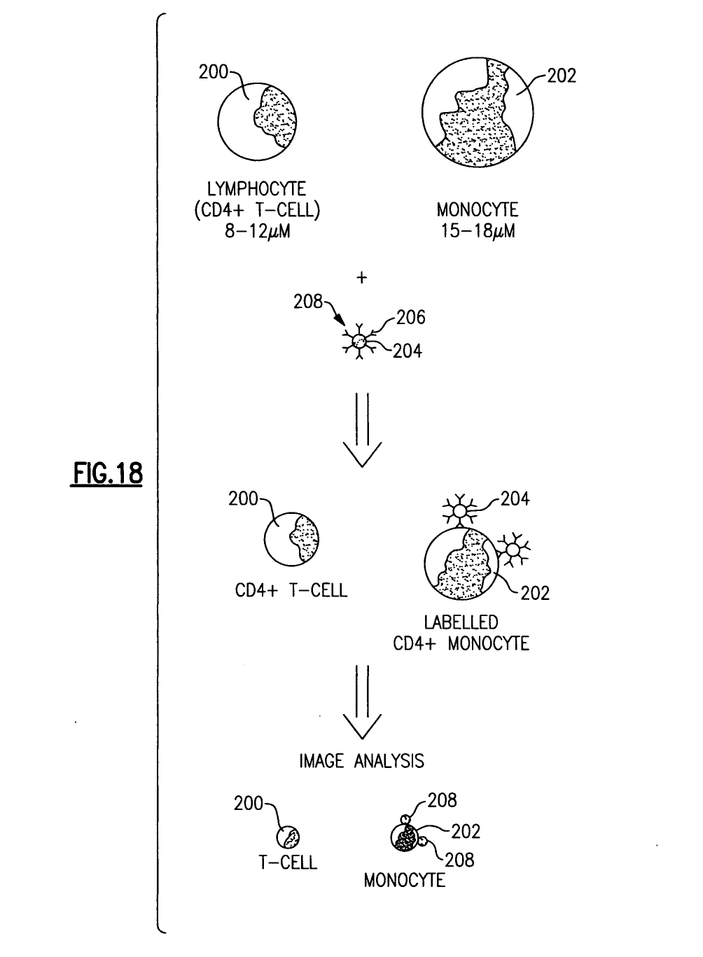

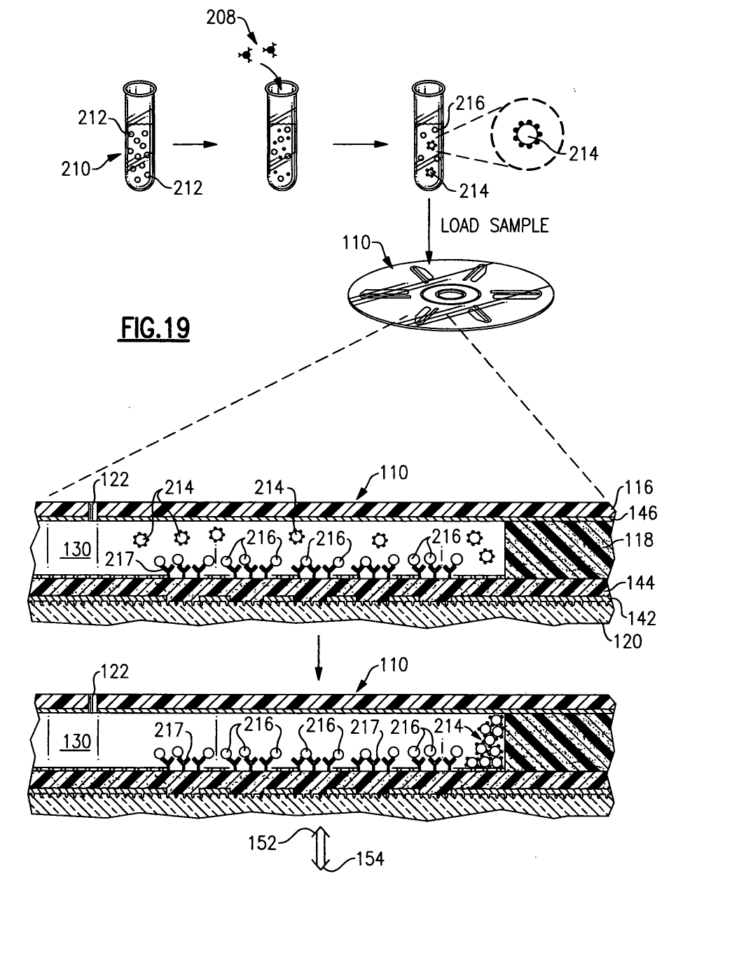

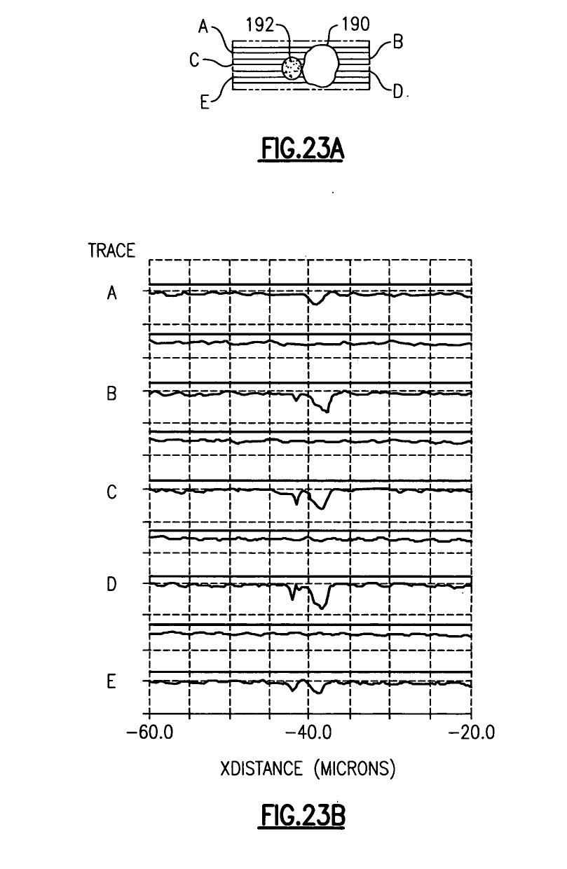

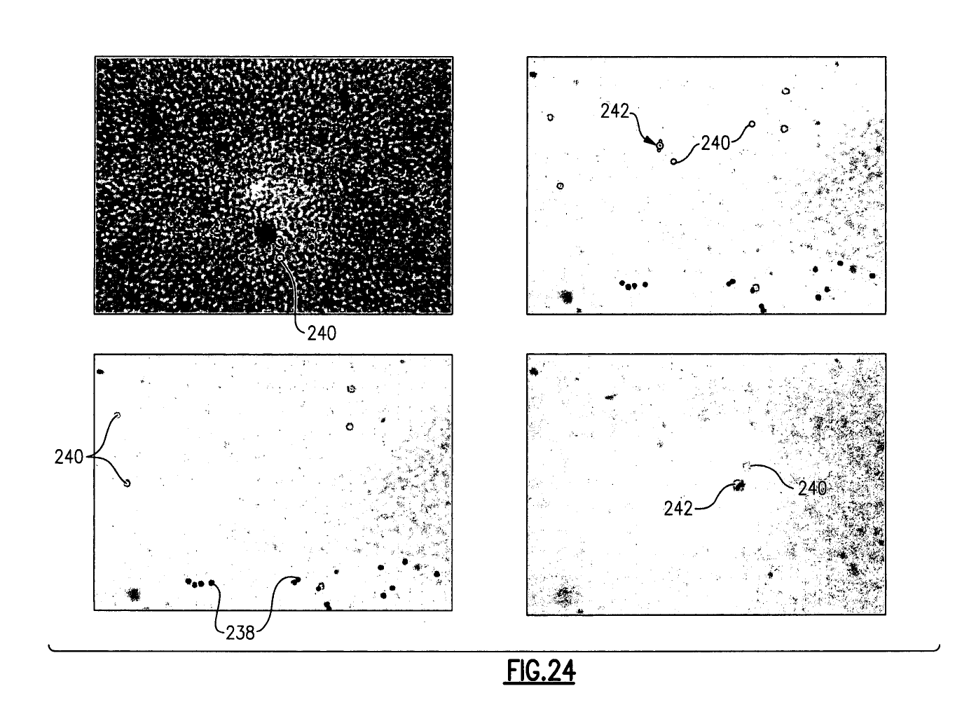

As described below, by using a bead as the reporter agent to label a cell, one can differentiate, for example, target CD4+ T cells from unwanted CD4+ monocytes, captured using anti-CD4 antibody capture agents. This is achieved by labelling or tagging antigenic epitopes, other than the CD4 epitopes, on the monocytes. For example, the monocytes may be tagged with beads attached to CD14 antibodies which are specific for monocytes. Once the monocytes are tagged with anti-CD14 beads, image analysis is carried out using the optical bio-disc system to distinguish between CD4+ monocyte cells, with one or more beads bound to its CD14 surface antigen (as described below in FIG. 23), and the CD4+ T cells which are free of beads because of the absence of CD14 antigens on its surface. Thus allowing the differentiation between CD4 T cells and monocytes in a MNC or blood sample as depicted in FIG. 18.

With continuing reference to FIG. 18, there is illustrated a lymphocyte 200 and a monocyte 202, both having CD4 antigens on their surface. Monocytes also have CD14 antigens on their surface while lymphocytes do not. Thus monocytes may be differentiated from lymphocytes by tagging them with beads 204 having a signal antibody 206, which is anti-CD14 antibody in this case, attached thereto. The beads are preferably large enough to be detected by the interrogation beam 152 (FIG. 10) of the disc reader 112 and smaller than the cells to be tagged. The preferred size of the beads is around 0.5 to 5 um in diameter. The reporter complex 208 will thus bind only to monocytes 202 when placed in a suspension of cells containing CD4+lymphocytes 200 and CD4+monocytes 202 as illustrated. When these CD4+ cells are captured on the capture spot 140 having anti-CD4 capture antibodies attached thereto and analysed using the optical disc reader 112 (FIG. 1), the cells may be differentiated by the resulting signature traces as described below in conjunction with FIGS. 23A and 23B.

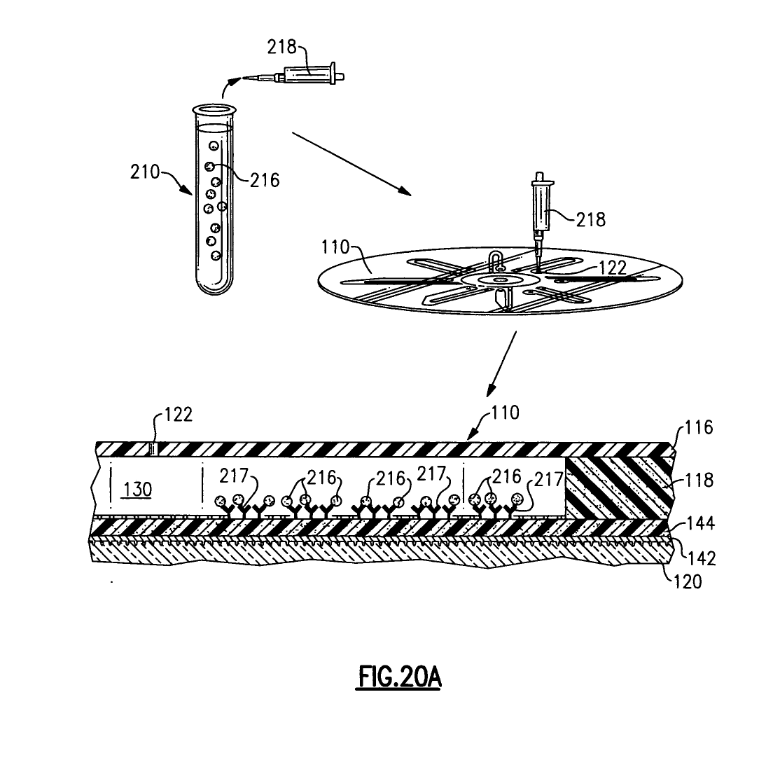

Referring now to FIG. 19, there is illustrated an embodiment of the present invention depicting the use of beads to isolate or remove unwanted cells from a blood sample. The unwanted or contaminant cells are removed by blocking antigens on the surface of the unwanted cells using beads thereby preventing binding of these blocked cells to a capture probe on the disc. In the example shown in FIG. 19, a sample 210 containing cells of various types 212 including CD4+ cells, CD8+ cells and natural killer (NK) cells is processed for analysis. All of the cells 212 have CD3 markers on their surface. CD4+ and CD8+ cells are the target cells of interest 216 while the NK cells 214 are the unwanted cells. A common antibody, anti-CD3, is used to capture the target cells. The NK cells contain CD56 antigens on their surface while the other cells in the sample do not. As shown, antibody coated beads 208 are mixed in with the sample 210. The beads 208 are coated with anti-CD56 antibodies. Beads 208 then bind to the CD56 antigens on the surface of the NK cells 214 rosetting or surrounding the NK cells. The assay solution containing the CD4+, CD8+ and rosetted NK cells 214 is then loaded into the fluidic chamber 130 in the bio-disc 110 as depicted. The CD4+ and CD8+target cells 216 then bind to anti-CD3 capture agents 217 on the surface of the disc. The beads on the surface of the NK cells 214 prevent the binding of the NK cells 214 to the anti-CD3 capture antibody 217 by blocking the CD3 antigen epitopes on the NK cells. The unbound NK cells may then be removed by washing or centrifugation as illustrated. The captured cells are then analysed using the optical bio-disc system (FIG. 10) by scanning the incident beam 152 (FIG. 10), which interacts with the captured cells, through the target zones and analysing the return beam 154 to determine the relative amount of target cells in the sample. Alternatively, if the transmissive type optical disc is used, the transmitted beam 156 (FIG. 10) may be analysed to determine the number of captured cells. The optical bio-disc illustrated in FIG. 19, includes the disc components described above in conjunction with FIGS. 2 to 9 including the flow channel 130, cap portion 116, reflective surface 156, adhesive member 118, active layer 144, reflective layer 142 and substrate 120. As mentioned above, the transmissive type optical bio-disc (FIGS. 8 and 9) may also be used wherein the reflective layer 146 is removed and layer 142 is semi-reflective to allow the incident beam 152 to pass through the disc which allows detection and analysis of the transmitted beam 156 using a top detector 158 as described above in conjunction with FIGS. 10, 12 and 16.

In and alternative embodiment to the method described above in conjunction with FIG. 19, larger, heavier, and/or magnetic beads are attached to the unwanted cells. The target cells, for example may be CD4+ cells while the unwanted cells are CD4+ monocytes. The capture antibody used in this example may be anti-CD4 antigen. Since both cells types have CD4 antigens on their cell surface and monocytes have a unique CD14 surface marker, monocytes are removed from the sample using magnetic beads, which have greater mass than non-magnetic beads of the same size, coated with anti-CD14 antibodies. The greater mass allows for easy removal of the unwanted beads by centrifugation. The beads may be mixed with the sample solution prior to or after loading the sample into the disc. The beads are then allowed to bind to the surface CD14 antigens on the monocytes. Since magnetic particles are relatively heavy, the monocytes may be separated from the cells of interest by centrifugation using the bio-disc drive 112, or by using the magnetic properties of the beads in conjunction with a magnetic separator or a magneto-optical disc system. Further details relating to other aspects associated with magneto-optical disc systems are disclosed in, for example, commonly assigned co-pending U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” filed Mar. 14, 2002; and U.S. patent application Ser. No. 10/307,263 entitled “Magneto-Optical Bio-Discs and Systems Including Related Methods” filed Nov. 27, 2002, all of which are incorporated herein by reference in their entirety.

Referring next to FIGS. 20A and 20B, there is shown a non-limiting example of an alternative embodiment, of the present invention, to that described in conjunction with FIG. 19. In this embodiment, beads having different physical properties are used to identify various cell types captured, by a common surface marker, in one or more target or capture zones. As shown in FIG. 20A, a sample 210 containing cells of interest 216 is loaded into the bio-disc 110 using a pipette 218. In this example, leukocytes are the cells of interest which are captured by anti-CD45 capture antibodies 217 on the surface of the disc. The capture antibodies 217 bind the common CD45 leukocyte surface antigen. With reference now to FIG. 20B, different groups of beads 220 are then loaded into the disc 110 using pipette 218. Each group of beads has different physical characteristics, preferably distinguishable using the disc reader 112, and different antibodies attached thereto. The antibodies attached to each group of beads will have affinity to a specific antigen on the cell of interest, for example, a group of transparent beads 221 having antibodies to either CD3, CD19, or CD56 to tag lymphocytes 224, a group of opaque beads 223 having antibodies to CD14 attached thereto to tag monocytes 222, and a group of semi-transparent beads 225 having antibodies to CD116 to tag eosinophils 226. After allowing sufficient time for beads to bind to their respective targets, the unbound beads 227 are removed by centrifugation or washing. The different cell types may then be quantitated based on the physical characteristics of the beads bound thereto using the optical disc reader. Since the transmissive type disc is used in this example, as illustrated, the bead-cells complexes are analyzed using the optical bio-disc system (FIG. 10) by scanning the incident beam 152 (FIG. 10), which interacts with the bead-cell complexes, through the target zones, detecting the transmitted beam 156 using a photo detector and analyzing the detected beam to determine the relative amount each respective target cell type in the sample. The reflective type disc may also be used for this analysis as described above.

Alternatively, one or more groups of beads or reporters may bind to different surface antigens on the same cell type. This cell type may then be quantitated and distinguished from the other cell types by determining the absence or presence of one or more types of reporters bound thereto. This adds specificity to the assay since multiple parameters are used to identify as single cell type. Further details relating to other aspects associated with methods for detecting and quantitating various cell types using the optical disc is disclosed in, for example, commonly assigned U.S. Provisional Patent Application Ser. No. 60/382,944 entitled “Methods and Apparatus for Use in Detection and Quantitation of Cell Populations and Use of Optical Bio-Disc for Performing Same” filed May 24, 2002, which is incorporated herein by reference in its entirety.

Micro-particles or beads may also be used to label or tag cells from tissues, and microorganisms such as viruses and bacteria to facilitate identification, differentiation, and quantitation. FIG. 21 shows an example of tagging E. coli 234 with a capture bead 204 having at least one biotin 232 and a capture antibody 206 conjugated thereto. Antibody 206 has a specific affinity to an antigen on E. coli. A bead-bacteria complex 237 is formed when the beads are mixed with a sample containing E. coli, as illustrated. The complex 237 and unbound beads 204 are then captured on a capture zone in the disc using streptavidin 236. The disc is then analysed for the presence and amount of bead-bacteria complexes as described above.

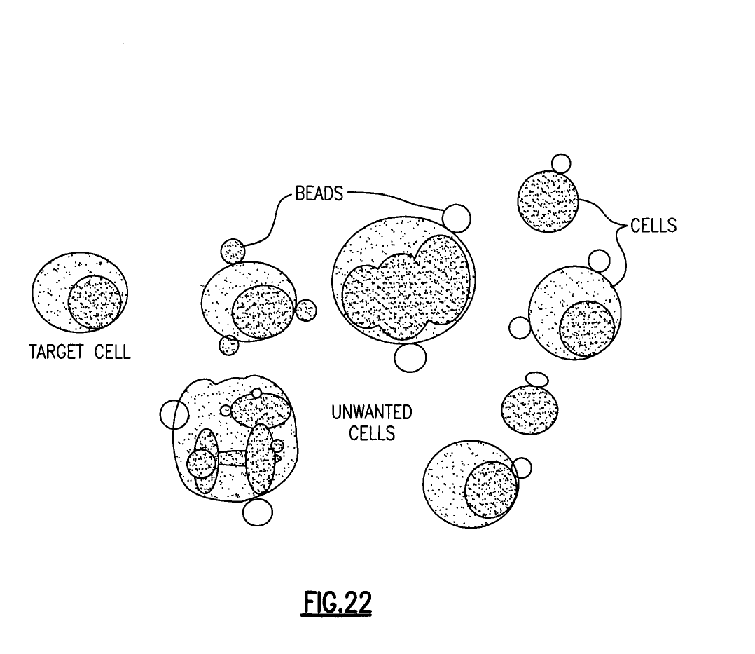

The next figure, FIG. 22, is an illustration of tagging of unwanted cells using beads having antibodies with affinity to antigens on the unwanted cells. The target cells are thus untagged and are isolated or quantitated using the optical bio-disc system as described above.

With reference now to FIG. 23A, there is shown a graphical representation of a 1 micron reporter bead and a 5 micron cell linked together in a bead-cell complex positioned relative to the tracks A-E of an optical bio-disc according to the present invention.

Referring next to FIG. 23B, there are illustrated a series of signature traces, from tracks A-E, derived from the bead and cell of FIG. 23A utilizing a detected signal from the optical drive according to the present invention. These graphs represent the detected transmitted beam 156. As shown, the signatures for the 1 micron reporter bead 190 are sufficiently different from those for the 5 micron cell 192 such that the bead-cell complex can be detected and discriminated from single cells. A sufficient deflection of the trace signal from the detected return beam as it passes through a bead or cell is referred to as an event. The relative proximity of the events from the reporter and cell indicates the presence or absence the bead cell complex. As shown, the traces for the reporter and the cell are right next to each other indicating they are joined in a complex.

Alternatively, other detection methods may be used to identify and quantify various cell types in a cell suspension. For example, reporter beads can be fluorescent or phosphorescent. Detection of these reporters can be carried out in fluorescent or phosphorescent type optical disc readers. Other signal detection methods are described, for example, in commonly assigned co-pending U.S. patent application Ser. No. 10/008,156 entitled “Disc Drive System and Methods for Use with Bio-Discs” filed Nov. 9, 2001, which is expressly incorporated by reference; U.S. Provisional Application Ser. No. 60/270,095 filed Feb. 20, 2001 and No. 60/292,108, filed May 18, 2001; and the above referenced U.S. patent application Ser. No. 10/043,688 entitled “Optical Disc Analysis System Including Related Methods For Biological and Medical Imaging” filed Jan. 10, 2002.

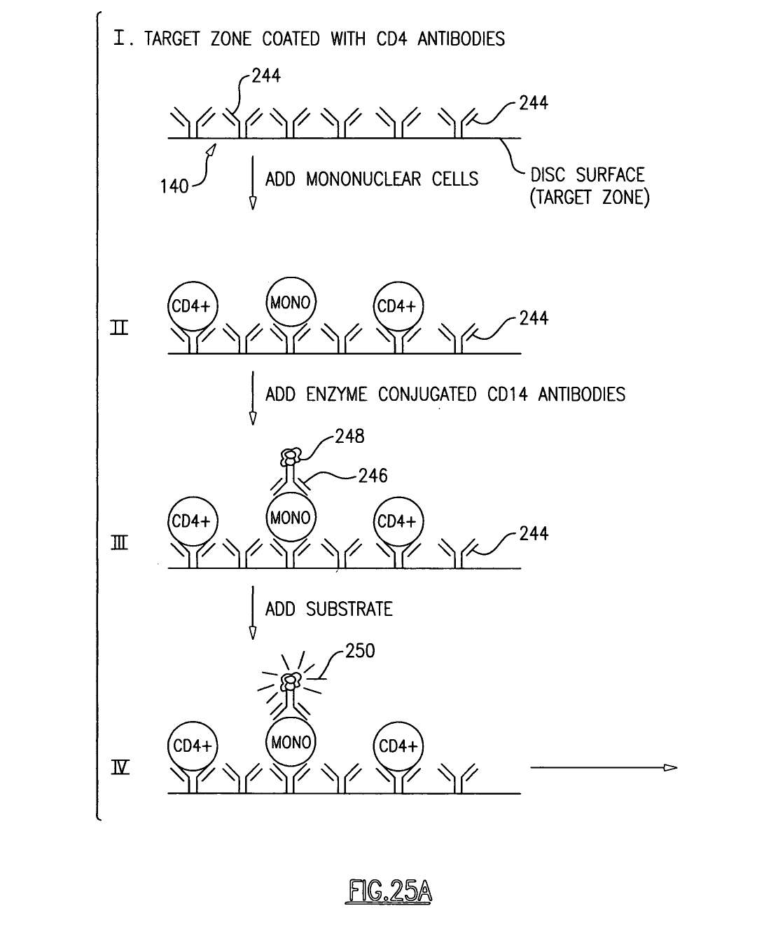

In FIG. 24, there are illustrated micrographs showing unattached beads 238, single cells 240, and bead-cell complexes 242. The beads used in this experiment were 4.5 um magnetic beads coated with anti-CD2 antibodies. Cells having CD2 antigens are thus labeled or tagged with the beads forming a bead-cell complex while non-CD2+ cells remain single as shown. Details relating to this experiment are discussed below in conjunction with Example 8.

Use of Insoluble Enzyme Products to Differentiate Target Cells

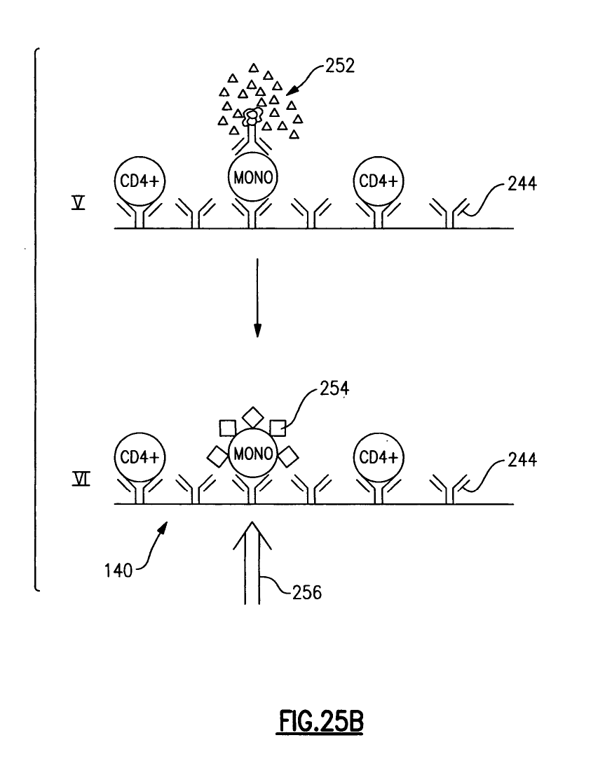

Referring now to FIGS. 25A and 25B, there is illustrated a pictorial representation of another embodiment of a method of the present invention for differentiating unwanted cells from target cells using detectable precipitates from an enzyme reaction. A magnified view of the target zone 140 within the fluidic circuit 130 is illustrated in FIG. 25. As would be apparent to one of skill in the art given the present disclosure, either the reflective or transmissive type discs may be used in this analysis. In the example depicted in FIGS. 25A and 25B, CD4 capture antibodies 244 are deposited on the target or capture zone 140 on the optical bio-disc 110 (Step I). In the next step, Step II, a sample containing mononuclear cells (MNC) is then introduced into the target zone. The cells having CD4 surface antigens will then bind to the CD4 capture antibodies 244 on the target zone. These cells include CD4+ cells and monocytes. The unbound cells are then washed away or spun off the target zone as described above in conjunction with FIGS. 19 to 21. After removing the unbound cells, a solution containing an enzyme 248 conjugated to a CD14 antibody 246 (reporter agent) is introduced to the target zone as shown in Step III. Since only monocytes contain CD14 antigens, the enzyme conjugated antibodies only bind to any monocyte bound to the target zone as illustrated. The target zone is then washed to remove unbound enzyme or reporter agent and an enzyme substrate is introduced to the target zone in Step IV. Once the substrate comes in contact with the enzyme, an enzyme-substrate reaction 250 occurs which produces a detectable product 252, shown in Step V. The detectable product 252 is preferably an insoluble product that forms precipitates 254 on the cell surface, as illustrated in Step VI. An interrogation beam of electromagnetic radiation 256 is then scanned through the target zone and the return beam or transmitted beam, depending on the type of disc used, is analysed for the presence and absence of precipitate labelled cells. The labelled or tagged and unlabelled or untagged cells are then quantitated. This allows for the specific detection, differentiation and quantitation of CD4+ cells and monocytes as shown in FIG. 25B. The enzymes that may be used in this embodiment include, but not limited to, horse radish peroxidase (HRP) and alkaline phosphatase (AP). The substrates that may be used in conjunction with these enzymes may be, for example, chosen from the group consisting of TMB (3,3′, 5,5′-tetramethyl benzidine), DAB (3,3′-Diaminobenzidine), ABTS (2,2′-Azino-bis(3-ethylbenzthiazoline-6-sulphonic acid)), AEC (3-Amino-9-ethylcarbazol), NBT (Nitro Blue Tetrazolium), CN/DAB (4-chloronaphthol/3,3′-diaminobenzidine, tetrahydrochloride), (4-CN) 4-chloro-1-napthol, and other compatible substrates that enables the enzyme to catalyse reactions that result in products detectable by an optical disc reader.

Experimental Details

While this invention has been described in detail with reference to the drawing figures, certain examples and further illustrations of the invention are presented below.

### EXAMPLE 1

FIG. 17 illustrates a pictorial flow chart showing the preparation of a sample, use of a bio-disc, and the provision of results. The details of the following example such as the individual time duration of process steps, rotation rates, and other details are more particular than those described above with reference to FIG. 17. The basic steps of the present example are, nonetheless, similar to those described above.

A. Disc Manufacturing Including Substrate Preparation and Chemistry Deposition

In this example, a reflective disc or transmissive disc substrate 120 (FIGS. 2 and 5, respectively) is cleaned using an air gun to remove any dust particles. The disc is rinsed twice with iso-propanol, using a spin coater. A 2% polystyrene is spin coated on the disc to give a very thick coating throughout.

The chemistry is then deposited. One embodiment includes a three step deposition protocol that incubates: streptavidin, incubated for 30 minutes; biotinylated first antibody incubated for 60 minutes; and a second capture antibody incubated for 30 minutes. The first antibody can be raised in a first species (e.g., sheep) against a type of immunoglobulin (e.g., IgG, IgE, IgM) of a second species (e.g., mouse). The second capture antibody is raised in the second species against a specific cell surface antigen (e.g., CD4, CD8). The steps are done at room temperature in a humidity chamber using washing and drying steps between depositions.

A 1 μl ratio of 1 mg/ml streptavidin in phosphate buffered saline is layered over each window and incubated for 30 minutes. Excess streptavidin is rinsed off using distilled water and the disc is dried. Equal volumes of biotinylated anti-mouse IgG (125 μg/ml in PBS) and activated dextran aldehyde (200 μg/ml) are combined. Dextran aldehyde (DCHO)-biotinylated anti-mouse IgG is layered over streptavidin in each capture window and incubated for 60 minutes or overnight in refrigerator. Excess reagent is rinsed and the disc is spun dry.

B. Disc Assembly

The disc is assembled using an adhesive layer that may, for example, be 25, 50, or 100 microns thick (channel layer 118 in FIGS. 2 and 5), with a stamped out portion, such as a U-shape or “e-rad” channel, to create a fluidic channel, and a clear cap 116 (FIG. 5, for use with a transmissive disc with a top detector) or a cap 116 with a reflective layer 142 located over the capture zones (FIG. 2, for use with a reflective disc with a bottom detector).

In one embodiment, the disc is a forward Wobble Set FDL21:13707 or FDL21:1270 CD-R disc coated with 300 nm of gold as the encoded information layer. On a reflective disc, viewing windows of size 2×1 mm oval are etched out of the reflective layer by known lithography techniques. In some designs of transmissive disc, no separate viewing windows are etched, and the entire disc is available for use. In this particular example, the channel layer is formed from Fraylock adhesive DBL 201 Rev C₃M94661. The cover is a clear disc with 48 sample inlets each with a diameter of 0.040 inches located equidistantly at radius 26 mm. The data disc is scanned and read with the software at speed 4× and sample rate 2.67 MHz using CD4/CD8 counting software.

C. Disc Leak Check

Because blood is being analyzed, the disc can be leak checked first to make sure none of the chambers leak during spinning of the disc with the sample in situ. Each channel is filled with a blocking agent, such as StabilGuard and PBS-Tween. The block is for at least 1 hour. The disc is spun at 5000 rpm for 5 minutes to leak proof and check disc stability. After checking for leaks, the disc is placed in a vacuum chamber for 24 hours. After vacuuming for 24 hours, discs are placed in a vacuum pouch and stored in refrigerator until use.

D. Sample Collection, Preparation, and Application to Disc

The following section is directed to sample processing steps which are generally shown in FIG. 17A. Mononuclear cells (MNC) are purified by a density gradient centrifugation method, e.g., using a Becton Dickinson CPT Vacutainer. Blood (4-8 ml) is collected directly into a 4 or 8 ml EDTA containing CPT Vacutainer. The tubes are centrifuged at 1500 to 1800×g in a biohazard centrifuge with horizontal rotor and swing out buckets for 25 minutes at room temperature. The blood is preferably used within two hours of collection. After centrifugation, plasma overlying the mononuclear cell fraction is removed, leaving about 2 mm of plasma above an MNC layer. MNC are collected and washed with PBS. Cells are pelleted by centrifuge at 300×g for 10 minutes at room temperature. The supernatant is removed and the pellet containing the MNC is resuspended in PBS by tapping the tube gently. One more washes are done at 300×g for 10 minutes each at room temperature to remove platelets. The final pellet is resuspended to a cell count of 10,000 cells/pi. An 18 μl volume of the MNC is introduced to one or more the analysis chamber or channel, incubated for 15 minutes at room temperature with the disc stationary. The channels are sealed. The disc is then spun at 300 rpm for 3 to 4 minutes using a disc drive. The disc is preferably scanned and read with the software at speed 4× and sample rate 2.67 MHz.

If a blood sample cannot be processed immediately, mononuclear cells after the first centrifugation can be resuspended in plasma by gently inverting the CPT tube several times and stored for up to 24 hours at room temperature. Within 24 hours, the cells in the plasma can be collected and washed as described above.

E. CD4/CD8 Assay Format

The assay in this example is a generic homogeneous solid phase cell capture assay for the rapid determination of absolute number of CD4+ and CD8+T-lymphocyte populations and ratio of CD4+/CD8+lymphocytes in blood samples. The test, which is run within a small chamber incorporated into a CD-ROM, determines the number of CD4+, CD8+, CD2+, CD3+ and CD45+cells captured by the specific antibodies on the capture zones in 7 μl of mononuclear cells (MNC) isolated from whole blood. The test is based upon the principle of localized cell capture on specific locations on the disc. Several specific cell capture zones are created on the disc by localized application of capture chemistries based upon monoclonal or polyclonal antibodies to particular blood cell surface antigens. Upon flooding the chamber with the MNC blood (30,000 cells/μl), cells expressing antigens CD4, CD8, CD2, CD3 and CD45 are captured in the capture zones on the disc. Also incorporated within the bar code are defined negative control areas.

F. On-Disc Analysis

MNC cells, prepared in step D above (18 μl in PBS), are injected into the disc chamber, and inlet and outlet ports of the chamber are sealed. The disc is incubated for 15 minutes at room temperature, and then scanned using a 780 nm laser in an optical drive with a top detector to image the capture field as described above.

Software is encoded on the disc to instruct the drive to automatically perform the following acts: (a) centrifuge the disc to spin off excess unbound cells in one or more stages, (b) image specific capture windows, and (c) process data including counting the specifically-captured cells in each capture zone and deriving the ratio of CD4/CD8 (or which ever ratio is programmed to be determined).

During the processing step, the software reads across each capture zone image and marks cells as it encounters them. For example, following estimation of number of CD4+ and CD8+cells, the software calculates the ratio of CD4+/CD8+cells and displays both the absolute numbers of cells in CD4+, CD8+, CD3+ and CD45+capture zones per microliter of whole blood and also the CD4+/CD8+ratio. The entire process takes about 12 minutes from inserting the disc into the optical drive to obtaining the numbers and ratios.

G. Reagents Used

Streptavidin (Sigma, cat. # S4762): Add de-ionized water to make a 5 mg/ml solution, aliquot and store at −30° C. To use, add Tris buffer for a final concentration of 1 mg/ml.

Positive control: CD45 (Sigma, Lot # 038H4892, cat # C7556). Store at 2-8° C.

Secondary capture antibody: Biotinylated anti-mouse IgG (raised in sheep, Vector laboratories, lot # L0602, Catalog # BA-9200) Stock solution 1.5 mg/ml made in distilled water. Working b-IgG solution 125 μg/ml in 0.1M PBS. Store at 2-8° C. May be kept at −30° C. for long term storage.

Aldehyde activated Dextran (Pierce, lot # 97111761, cat # 1856167). Stock solution stock solution 5 mg/ml in PBS, store at 2-8° C.

Primary capture antibody: CD4 (DAKO, cat # M0716), CD8 (DAKO, cat # M0707), CD2 (DAKO, cat # M720), CD45 (DAKO, cat # M0701), CD14 (DAKO, cat # M825), and CD3 (DAKO, cat # M7193). Store at 2-8° C.

Negative control: Mouse IgG1 (DAKO, cat # X0931). Store at 2-8° C.

Phosphate Buffered Saline (PBS), pH 7.4 (Life Technologies/GIBCO BRL, cat. # 10010-023) or equivalent. Store at room temperature

Isopropyl alcohol, 90-100%

H. RBC Lysing Protocol

Ammonium Chloride Lysing Buffer

A 1× stock of ammonium chloride lysing buffer should be stored at 2 to 8° C. Comprised of 0.155M NH₄Cl, 10 mM KHCO₃, and 0.1 mM disodium EDTA; pH7.3 to 7.4. Store at 2-8° C. Bring to room temperature prior to use.

Procedure
1. For every 100 μl of blood add 2 ml of lysing buffer. (It is preferable to do this procedure in a biohazard hood.)
2. Vortex and incubate for 15 minutes at room temperature.

3. Centrifuge the blood at 500×g for 5 minutes at room temperature, using the centrifuge in the biohazard hood.

4. Remove supernatant and wash cells with 2% FCS or FBS in PBS. Centrifuge cells.

5. Calculate the total amount of WBCs and make the final concentration of WBCs 10,000 cells/pi for sample injection.

### EXAMPLE 2

### Mononuclear Cells Separation Procedure

Use Becton and Dickinson Vacutainer CPT (BD catalog # 362760 for 4 ml, #362761 for 8 ml) cell preparation tubes with sodium citrate. Do procedure in biohazard hood following all biohazard precautions. Steps:
1. Collect blood directly into a 4 or 8 ml EDTA containing CPT Vacutainer. If the blood sample is already in an anticoagulant, pour off the EDTA in the Vacutainer first and then pour 6-8 ml of blood sample into the CPT tube.
2. Centrifuge the tube at 1500 to 1800×g in a biohazard centrifuge with horizontal rotor and swing out buckets for 25 minutes at room temperature. For best results, the blood should be centrifuged within two hours of collection. However, blood older then 2 hours may be centrifuged with a decrease in MNC number and increase in RBC contamination.
3. After centrifugation, remove the plasma leaving about 2 mm of plasma above the MNC layer. Collect and transfer the whitish mononuclear layer into a 15 ml conical centrifuge tube.
4. Add 10-15 mls with PBS to MNC layer, gently mix the cells by inverting the centrifuge tube several times.
5. Wash cells by centrifuge at 200×g for 10 minutes at room temperature in biohazard centrifuge.
6. Remove supernatant. Resuspend cells by tapping the tube gently.
7. Wash one more time in 10 ml of PBS. Centrifuge at 200-300×g for 10 minutes at room temperature to remove platelets.
8. Remove supernatant and resuspend pellet in 50 ul PBS.
9. Estimate cell counts in the sample. Run CBC or dilute 2 ul of cells to 18 ul of trypan blue, gently mix and count cells with a hemocytometer. Make up the sample to a final cell count of 10,000 cells/ul for analysis.
10. If the cells cannot be processed immediately, resuspend mononuclear cells after the first centrifugation (step 2 above) in the separated plasma by gently inverting the CPT tube several times and store for up to 24 hours at room temperature. Within 24 hours, collect the cells in the plasma and continue with the washes as described above.
Total cell counts per ul=number of cells in 25 small squares×(times) 100.

### EXAMPLE 3

### Isolation of MNCs from Whole Blood Using Histopaque-1077

1 ml of Histopaque-1077 was placed in a 15 ml centrifuge tube and 1 ml of whole blood is gently layered over that. Then centrifuged at 400×g for 30 min at room temperature. The mixture was aspirated carefully with a pasture pipette and the opaque interface transferred to centrifuged tube. Then 10 ml of PBS was added to the centrifuge tube. The solution was then centrifuged at 250×g for 10 min. The supernatant was decanted and the cell pellet was resuspended in 10.0 ml PBS and spun at 250×g for 10 min. The cells were then washed one more time by resuspending the pellet in 10 ml PBS and spinning at 250×g. The final cell pellet was resuspended in 0.5 ml PBS.

### EXAMPLE 4

### CD4+Cell Isolation with Dynal Beads

A. Materials

1. Cold PBS/2% FBS, pH7.4
2. PBS/0.5% BSA, pH 7.4
3. CD4 Positive Dynal Isolation Kit
4. Dynal MPC, Dynal mixer, Centrifuge, Polypropylene tubes
   B. Procedure

Run a CBC and determine the number of beads per cell needed (4-10 beads/cell). Add 1 ml of cold PBS/2% FBS to desired amount of beads (1×10⁷ beads/72 ul) and resuspend. Place tube in Dynal MPC for 30 seconds and pipette off the supernatant. Resuspend the washed beads to the original volume. Add the desired amount of beads to the cells. Incubate at 2-8° C. for 20 minutes in the Dynal mixer set to 11. Isolate the rosetted cells in the Dynal MPC for 2 minutes. Pipette off the supernatant. Wash the rosetted cells 4× in PBS/2% FBS. Resuspend the rosettes in 200-400 ul of PBS/2% FBS. Add 10 ul Detach-a-Bead per 100 ul cell suspension. Incubate at RT for 60 minutes in the Dynal mixer set to 11. Isolate the beads in the Dynal MPC for 2 minutes. Transfer and save the supernatant. Wash the beads 2-3 times in 500 ul PBS/2% FBS to obtain residual cells. Wash the collected cells in 400 ul PBS/0.5% BSA. Run CBC to determine isolated cell concentration.

### EXAMPLE 5

### Disc Preparation and Chemistry Deposition (With Streptavidin)

A. Disc Manufacturing Including Substrate Preparation and Chemistry Deposition

In this example, a transmissive disc substrate was cleaned with an air gun to remove dust. The disc was then mounted in the spin coater and rinsed twice with a steady stream of iso-propanol. Next, a polystyrene solution with 2% polystyrene dissolved in 310 ml of toluene and 65 ml of iso-propanol was evenly coated onto the disc.

For the streptavidin deposition, streptavidin stock solution was diluted to 1 mg/ml in PBS. Using manual pin deposition, approximately 1 ul of the streptavidin was deposited in each capture zone on the disc. The disc was incubated in a humidity chamber for 30 minutes. Then excess unbound streptavidin was rinsed off the capture zones with D. I. water and the disc was spun dried.

For the secondary antibody deposition, a fresh solution of activated dextran aldehyde (200 ug/ml in PBS) was combined with an equal volume of the Vector IgG (125 ug/ml in PBS). Using manual pin deposition, approximately 1 ul of the IgG+DCHO complex was deposited (layered on top of the streptavidin layer) in each capture zone on the disc. The disc was incubated in a humidity chamber for 60 minutes. Excess antibody was rinsed off with D. I. water and the disc was spun dry.

For the primary antibody, DAKO CD4 was diluted to 50 ug/ml in PBS, DAKO CD8 was diluted to 25 ug/ml in PBS, and DAKO CD45 was diluted to 145 ug/ml in PBS. Using the manual pin applicator, deposited approximately 1 ul of each primary antibody on top of the bound secondary antibodies. The disc was then incubated in a humidity chamber for 30 minutes. The excess unbound antibody was removed by washing the capture zones with PBS and the disc was spun dried.

B. Disc Assembly

The cover disc used was a clear disc with a Fraylock adhesive channel layer attached thereto. Stamped into the adhesive were 4 U-shaped channels that created the fluidic circuits. The cover was placed onto the transmissive disc substrate so that the fluid channels were over the capture zones. Next, to secure the discs together, they were passed through a disc press 8 times.

C. Disc Leak Check, Blocking

Each fluid channel was filled with StableGuard and incubated for 1 hour. During the incubation, the disc was spun in the spin coater for 5 minutes at 5000 rpm. After the spin, the disc channels were checked for leaks. Next, the StableGuard was aspirated out of the channels, and the disc was placed under vacuum in a vacuum chamber overnight. The next morning, the disc was placed in a vacuum pouch and stored at 4° C.

### EXAMPLE 6

### Comparing Optical Bio-disc CD4/CD8 Ratios to FACs CD4/CD8 Ratios

A. Preparing of Clinical Blood Samples Using CPT Tubes

3 mis of clinical EDTA blood samples (Nos. 29, 30, 31, 32, 33, 34 and 35) were poured into individual CPT tubes that had the sodium citrate removed. The tubes were centrifuged for 25 minutes at 1500×g, at room temperature. After centrifugation, the upper plasmas, within 0.5 cm of the opaque MNC layers, were aspirated off. The remaining opaque MNC layers were transferred to clean 15 ml tubes and 12 ml of PBS were added to each.

Washing

The cell suspensions were then centrifuged at 250×g for 10 minutes. The supernatants were then aspirated off, and the cells resuspended in 14 ml of PBS. The suspensions were centrifuged at 250×g for 10 minutes again. Aspirated off the supernatants and resuspended each cell pellet with 200-175 ul of PBS. The cell concentrations for each sample were determined by counting the cells with a hemocytometer. Adjusted the final cell concentrations to 30,000 cells/ul for each sample.

B. Comparing Optical Bio-disc CD4/CD8 Ratios to FACs CD4/CD8 Ratios

Discs (Nos. 27a, 27b, 27c, 27d, 27e, 27f & 28) were prepared similar to example 5, using 25 um adhesive channels.

Each sample was injected into each corresponding disc as shown below in Table 2. After 30 minutes, the disc was centrifuged at 3000 rpm for 5 minutes. Light micrographs were then taken of the cells captured on the chemistry zones and the cells were counted. Each clinical sample also had a FACs analysis performed. The results from this experiment are shown below in Table 2.

TABLE 2
Comparing Optical Bio-disc CD4/CD8
Ratios to FACs CD4/CD8 Ratios

| | Sample | Disc | FACs |
|---|---|---|---|
| Disk # | # | CD4/CD8 | CD4/CD8 |
| 27a | 29 | 2.39 | 2.43 |
| 27b | 30 | 1.47 | 1.67 |
| 27c | 31 | 0.8 | 0.98 |
| 27d | 32 | 1.84 | 2.16 |
| 27e | 33 | 0.96 | 1.14 |
| 27f | 34 | 1.59 | 1.49 |
| 28 | 35 | 1.03 | 1.04 |

### EXAMPLE 7

### Disc Preparation and Chemistry Deposition

In this example, a transmissive disc substrate was cleaned with an air gun to remove dust. The disc was then mounted in the spin coater and rinsed twice with a steady stream of iso-propanol. Next, a polystyrene solution with 2% polystyrene dissolved in 310 ml of toluene and 65 mls of iso-propanol was evenly coated onto the disc.

For the secondary antibody deposition, a fresh solution of activated dextran aldehyde (200 ug/ml in PBS) was combined with an equal volume of the Vector IgG (125 ug/ml in PBS). Using manual pin deposition, approximately 1 ul of the IgG+DCHO complex was deposited in each capture zone on the disc. The disc was incubated in a humidity chamber for 60 minutes. Excess antibody was rinsed off with D. I. water and the disc was spun dry.

For the primary antibody, DAKO CD4 was diluted to 50 ug/ml in PBS, DAKO CD8 was diluted to 25 ug/ml in PBS, and DAKO CD45 was diluted to 145 ug/ml in PBS. Using the manual pin applicator, approximately 1 ul of each primary antibody was deposited on top of the absorbed secondary antibodies. Incubated in the humidity chamber for 30 minutes. Rinsed off the excess antibody with PBS and spun dry the disc.

### EXAMPLE 8

### Differentiating Target Cells from Unwanted Cells using Antibody Coated Beads

In this experiment, a transmissive disc was prepared as described above in Example 3.

A. Preparing Clinical Samples Using Ammonium Lysing Buffer

A 20 ml volume of 1× ammonium chloride lysing buffer was added to 1 ml of ACD blood sample. The samples were vortexed and incubated for 15 minutes at room temperature. Next, the samples were centrifuged at 500×g for 5 minutes at room temperature. We them removed the supernatant, and washed the cells with 3 mls of 2% BSA in PBS. The washed cells were then centrifuged at 500×g for 5 minutes. After centrifugation, the supernatant was discarded and the cells resuspended in 1 ml of 2% BSA in PBS. The final cell concentration in the suspension was 5,500 cells/ul.

B. Tagging and Analysis of Cells

Dynal Magnetic anti-CD2 [Dynabeads® CD2 (Prod No. 111.02)] beads were prepared by washing a stock solution of beads with 0.1% BSA in PBS three times. 72 ul of bead suspension was mixed in with the cells prepared in Section A above. The cell/bead suspension was then incubated at room temperature for 20 minutes to allow the beads to bind unwanted CD2+NK cells in the sample. After incubation, the suspension was loaded into the disc prepared above. The disc containing the suspension was then incubated for 15 minutes at room temperature to allow the CD4+ cells (T-lymphocytes and NK Cells) in the sample to bind to the anti-CD4 capture agents within the capture zone on the disc. The disc was then rotated at 1000 rpm for 5 minutes to remove unbound cells. Micrographs were then taken of the capture zones. These micrographs are shown in FIG. 24. Since only NK cells have the CD2 marker on its surface, NK cells 242 can be distiguished from T-lymphocytes 240 within the capture zone since the NK cells 242 are tagged with beads and the T-lymphocytes 240 are not, as Shown in FIG. 24.

Concluding Summary

This invention or different aspects thereof may be readily implemented in or adapted to many of the discs, assays, and systems disclosed in the following commonly assigned and co-pending patent applications: U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 16, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6, 2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; U.S. patent application Ser. No. 10/341,326 entitled “Method and Apparatus for Visualizing Data” filed Jan. 13, 2003; U.S. patent application Ser. No. 10/345,122 entitled “Methods and Apparatus for Extracting Data From an Optical Analysis Disc” filed on Jan. 14, 2003; U.S. patent application Ser. No. 10/347,155 entitled “Optical Discs Including Equi-Radial and/or Spiral Analysis Zones and Related Disc Drive Systems and Methods” filed on Jan. 15, 2003; U.S. patent application Ser. No. 10/347,119 entitled “Bio-Safe Dispenser and Optical Analysis Disc Assembly” filed Jan. 17, 2003; U.S. patent application Ser. No. 10/348,049 entitled “Multi-Purpose Optical Analysis Disc for Conducting Assays and Related Methods for Attaching Capture Agents” filed on Jan. 21, 2003; U.S. patent application Ser. No. 10/348,196 entitled “Processes for Manufacturing Optical Analysis Discs with Molded Microfluidic Structures and Discs Made According Thereto” filed on Jan. 21, 2003; U.S. patent application Ser. No. 10/351,604 entitled “Methods for Triggering Through Disc Grooves and Related Optical Analysis Discs and System” filed on Jan. 23, 2003; U.S. patent application Ser. No. 10/351,280 entitled “Bio-Safety Features for Optical Analysis Discs and Disc System Including Same” filed on Jan. 23, 2003; U.S. patent application Ser. No. 10/351,244 entitled “Manufacturing Processes for Making Optical Analysis Discs Including Successive Patterning Operations and Optical Discs Thereby Manufactured” filed on Jan. 24, 2003; U.S. patent application Ser. No. 10/353,777 entitled “Processes for Manufacturing Optical Analysis Discs with Molded Microfluidic Structures and Discs Made According Thereto” filed on Jan. 27, 2003; U.S. patent application Ser. No. 10/353,839 entitled “Method and Apparatus for Logical Triggering” filed on Jan. 28, 2003; U.S. patent application Ser. No. 10/356,666 entitled “Methods For Synthesis of Bio-Active Nanoparticles and Nanocapsules For Use in Optical Bio-Disc Assays and Disc Assembly Including Same” filed Jan. 30, 2003; U.S. patent application Ser. No. 10/370,272 entitled “Methods and an Apparatus for Multi-Use Mapping of an Optical Bio-Disc” filed Feb. 19, 2003; and U.S. Provisional Application Ser. No. 60/60/479,803 entitled “Fluidic Circuits for Sample Preparation Including Bio-Discs and Methods Relating Thereto” filed Jun. 19, 2003.

All patents, patent applications and other publications mentioned in this specification are incorporated in their entireties by reference as if fully repeated herein.

While this invention has been described in detail with reference to certain preferred embodiments, it should be appreciated that the present invention is not limited to those precise embodiments. For example, the methods described above may be used to differentiate targets other than blood cells. Other targets may include, but not limited to, viruses, tissue cells, bacteria, plant cells and microorganisms. Rather, in view of the present disclosure, which describes the current best mode for practising the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

1. A method of detecting and quantifying a cell type of interest among various types of cells, said method comprising the steps of:
providing a test sample having target cells, said target cells having a common surface marker, said target cells including a first cell type having a first surface marker and a second cell type having a second surface marker;
providing a bio-disc, said bio-disc comprising a substantially circular substrate having a center and an outer edge, a target zone disposed between said center and said outer edge, a plurality of capture antibodies bound to said substantially circular substrate within said target zone such that said plurality of capture antibodies are immobilized in said target zone, said plurality of capture antibodies having affinity for said common surface marker on said target cells;
depositing said test sample on said target zone;
allowing said target cells including said first and second type cells present in said test sample to bind with said plurality of capture antibodies through said common surface marker;
washing said target zone to remove any unbound cells;
providing a reporter agent having a signal antibody bound thereto, said signal antibody having specific affinity to said first surface marker;
depositing said reporter agent on said target zone;
allowing said signal antibody to bind to said first surface marker on said first cell type in said target zone to thereby tag said first cell type;
washing the target zone to remove any unbound reporter agent; and
scanning a beam of electromagnetic radiation over said target zone to thereby determine the number of tagged first cell type cells and untagged second cell type cells.

2. The method of claim 1 wherein said reporter agent is a bead having a detectable physical property.

3. The method of claim 2 wherein said detectable physical property is selected from the group consisting of color, size, texture, reflectivity, absorbance, mass, fluorescence, phosphorescence, and magnetic properties.

4. The method of claim 1 wherein said reporter agent is an enzyme.

5. The method of claim 4 further comprising the step of depositing onto said target zone an enzyme substrate that reacts with said enzyme to produce a detectable signal.

6. The method of claim 5 wherein said enzyme is selected from the group consisting of horseradish peroxidase and alkaline phosphatase.

7. The method of claim 6 wherein said enzyme substrate is selected from the group consisting of TMB (3,3′, 5,5′-tetramethyl benzidine), DAB (3,3′-Diaminobenzidine), ABTS (2,2′-Azino-bis(3-ethylbenzthiazoline-6-sulphonic acid)), AEC (3-Amino-9-ethylcarbazol), NBT (Nitro Blue Tetrazolium), CN/DAB (4-chloronaphthol/3,3′-diaminobenzidine, tetrahydrochloride), and 4-CN (4-chloro-1-napthol).

8. The method of claim 7 wherein said detectable signal is a precipitate, which adheres to said first cell type.

9. A method of making an optical bio-disc for detection of various types of cells in a sample, said method comprising the steps of:
providing a substrate having a center and an outer edge;
encoding information on an information layer associated with said substrate, said encoded information being readable by a disc drive assembly to control rotation of said disc;
forming a target zone on said substrate between said center and said outer edge;
applying an active layer in said target zone; and
depositing within said target zone, a plurality of capture antibodies, at least some of said capture antibodies attaching to said active layer to thereby become immobilized within said target zone.

10. The method of claim 9 further comprising the step of forming a flow channel in fluid communication with said target zone.

11. The method of claim 10 wherein said plurality of capture antibodies have affinity for a common surface antigen among said various types of cells.

12. The method of claim 11 further comprising the step of forming a chamber in fluid communication with the flow channel, said chamber having an inlet port.

13. A method of using the optical bio-disc made according to claim 12 to detect and quantify one or more types of cells, said method comprising the steps of:
loading a test sample having various types of cells into said flow channel through said inlet port to place said test sample in contact with said plurality of capture antibodies within said target zone, said various types of cells having said common surface antigen;
allowing said plurality of capture antibodies to bind with said common surface antigen to immobilize said various types of cells in said target zone;
rotating the optical bio-disc at a pre-determined speed and time to remove unbound cells from the target zone;
providing at least one group of signal antibodies, each antibody thereof having attached thereto a reporter agent unique for said group, each group of signal antibodies having affinity to one cell type among the various types of cells in said target zone;
loading said at least one group of signal antibodies into said flow channel through said inlet port to place said signal antibodies in contact with said various types of cells within said target zone;
allowing said at least one group of signal antibodies to bind to its respective cell type to thereby tag said respective cell type;
washing said target zone to remove unbound signal antibodies;
scanning a beam of electromagnetic radiation over said target zone; and
analysing a return beam from said target zone to thereby determine the number of untagged cells and cells tagged with said at least one group of signal antibodies.

14. A method of detecting and quantifying a cell type in a test sample having various types of cells, said method comprising the steps of:
providing a bio-disc including a substantially circular substrate having a center and an outer edge, a target zone disposed between the center and the outer edge, a plurality of capture antibodies bound to said substrate within said target zone such that said plurality of capture antibodies are immobilized in said target zone, said plurality of capture antibodies having affinity for a common antigen among said various types of cells;
depositing said test sample on said target zone;
allowing said plurality of capture antibodies to bind with said common antigen to immobilize said various types of cells in said target zone;
washing said target zone to remove unbound cells;
providing at least one group of signal antibodies, each antibody thereof having attached thereto a reporter agent unique for said group, each group of signal antibodies having affinity to one cell type among the various types of cells immobilized in said target zone;
allowing said at least one group of signal antibodies to bind to its respective cell type to thereby tag said respective cell type;
washing said target zone to remove unbound signal antibodies; and
scanning a beam of electromagnetic radiation over said target zone to thereby determine the number of untagged cells and cells tagged with said at least one group of signal antibodies.

15. An optical bio-disc for detecting a cell type among various cell types in a sample, said optical bio-disc comprising:
a substantially circular substrate having a center and an outer edge;
an active layer associated with said substrate;
a target zone disposed between said center and said outer edge; and
a plurality of capture antibodies bound to said active layer such that said antibodies are immobilized on said active layer in said target zone.

16. The optical bio-disc according to claim 15 wherein said active layer is selected from the group consisting of nitrocellulose, polystyrene, polycarbonate, polystyrene-co-maleic anhydride, and gold.

17. The optical bio-disc according to claim 16 wherein said substrate includes encoded information associated therewith, said encoded information being readable by a disc drive assembly to control rotation of said bio-disc.

18. The optical bio-disc according to claim 17 further comprising a reflective layer formed on a surface of said substrate.

19. The optical bio-disc according to claim 18 further comprising a flow channel in fluid communication with said target zone and an input site in fluid communication with said flow channel.

20. The optical bio-disc according to claim 19 further comprising an enzyme, wherein said enzyme, when exposed to an enzyme substrate, produces a signal detectable by an incident beam of electromagnetic radiation.

21. The optical bio-disc according to claim 19 wherein said plurality of capture antibodies have affinity to a common surface marker on cells.

22. A method of using the optical bio-disc of claim 21 for differentiating and quantifying a cell type of interest, said method comprising the steps of:
providing a sample containing said cell type of interest and a non-target cell type, both cell types having said common surface marker, said non-target cell type having a unique surface marker;
providing a tagging agent attached to an antibody having affinity to said unique surface marker on said non-target cell type;
mixing said tagging agent with said sample;
allowing said tagging agent to bind with said unique surface marker on said non-target cell type to thereby block said common surface marker on said non-target cell type thus preventing binding of said non-target cell type to said plurality of capture antibodies in said target zone;
loading said sample into said flow channel through said input site to place said sample in contact with said capture antibody in said target zone;
allowing said capture antibody to bind with said common surface marker on said cell type of interest;
rotating said optical bio-disc at a pre-determined speed and duration to remove unbound non-target cells from the target zone; and
scanning a beam of electromagnetic radiation over said target zone to determine the number of said cell type of interest in said target zone.

23. The method according to claim 22 wherein said sample are mononuclear cells including lymphocytes and monocytes.

24. The method according to claim 23 wherein said common surface marker is a CD4 antigen.

25. The method according to claim 24 wherein said unique surface marker is a CD14 antigen.

26. A method of identifying and quantifying various cell types using the optical bio-disc made according to claim 12, said method comprising the steps of:
providing a sample containing said various cell types having said common surface antigen, said various cell types including a first cell type having a first surface marker, a second cell type having a second surface marker and a third cell type having a third surface marker;
loading said sample into said flow channel through said inlet port to place said sample in contact with said plurality of capture antibodies within said target zone;
allowing said plurality of capture antibodies to bind with said common surface antigen to immobilize said various cell types in said target zone;
rotating said optical bio-disc at a pre-determined speed and time to remove unbound cells from the target zone;
providing a labeling solution containing different types of reporter agents, said different types of reporter agents including a first reporter agent having attached thereto a first signal antibody with affinity to said first surface marker, a second reporter agent having attached thereto a second signal antibody with affinity to said second surface marker and a third reporter agent having attached thereto a third signal antibody with affinity to said third surface marker;
loading said labeling solution into said flow channel through said inlet port to place said reporter agents in contact with said cells immobilized in said target zone;
allowing said first signal antibody to bind with said first surface marker on said first cell type in said target zone thereby labeling said first cell type with said first reporter agent;
allowing said second signal antibody to bind with said second surface marker on said second cell type in said target zone thereby labeling said second cell type with said second reporter agent;
allowing said third signal antibody to bind with said third surface marker on said third cell type in said target zone thereby labeling said third cell type with said third reporter agent;
rotating said optical bio-disc at a pre-determined speed and time to remove unbound reporter agents from the target zone; and
scanning a beam of electromagnetic radiation over said target zone to thereby determine the number of cells tagged with said first reporter agent, the number of cells tagged with said second reporter agent and the number of cells tagged with said third reporter agent.

27. The method according to claim 26 wherein said sample are mononuclear cells.

28. The method according to claim 27 wherein said common surface antigen is a CD45 antigen.

29. The method according to claim 28 wherein said plurality of capture antibodies are anti-CD45 antibodies.

30. The method according to claim 29 wherein said first cell type is a lymphocyte.

31. The method of claim 30 wherein said first surface marker on said first cell type is selected from the group consisting of CD3, CD19 and CD56 antigens.

32. The method of claim 31 wherein said first signal antibody is selected from the group consisting of anti-CD3 antibody, anti-CD19 antibody, and anti-CD 56 antibody.

33. The method of claim 29 wherein said second cell type is a monocyte.

34. The method of claim 33 wherein said second surface marker on said second cell type is a CD14 antigen.

35. The method of claim 34 wherein said second signal antibody is an anti-CD14 antibody.

36. The method of claim 29 wherein said third cell type is a eosinophil.

37. The method of claim 36 wherein said third surface marker on said third cell type is a CD116 antigen.

38. The method of claim 37 wherein said third signal antibody is an anti-CD 116 antibody.

39. A method of detecting and quantitating target cells in a test sample, said method comprising the steps of:
providing a bio-disc, said bio-disc comprising a substantially circular substrate having a center and an outer edge, an active layer associated with said substrate, a target zone disposed between said center and said outer edge, at least one capture antibody bound to said active layer such that said capture antibody is immobilized on said active layer in said target zone, said capture antibody having affinity to said target cell;
depositing said test sample on said target zone;
allowing any target cell present in said test sample to bind with said capture antibody;
washing said target zone to remove any unbound cells;
providing a plurality of signal antibodies, each signal antibody having an affinity agent that binds to a binding agent;
allowing said signal antibodies to bind to any target cell that have antigens recognized by said signal antibodies to thereby tag any target cell;
washing said target zone to remove any unbound signal antibodies;
providing a plurality of reporter agents, each reporter agent having one or more binding agents;
allowing said reporter agents to bind to any signal antibody bound to any target cell in said target zone;
washing said target zone to remove any unbound reporter agents; and
scanning a beam of electromagnetic radiation over said target zone and analysing a return beam to thereby determine the number of tagged and untagged cells.

40. The method of claim 39 wherein said reporter agent is a bead having a physical property detectable using an optical disc reader.

41. The method of claim 40 wherein said physical property is selected from the group consisting of color, size, texture, reflectivity, absorbance, mass, fluorescence, phosphorescence, and magnetic properties.

42. The method of claim 39 wherein said reporter agent is an enzyme.

43. The method of claim 42 further comprising the step of depositing onto said target zone at least one enzyme substrate that reacts with said enzyme to produce a detectable signal.

44. The method of claim 39 wherein said affinity agent is biotin.

45. The method of claim 42 wherein said enzyme is conjugated with a binding agent that binds to said affinity agent.

46. The method of claim 45 wherein said binding agent is selected from the group consisting of streptavidin and neutravidin.

47. The method of claim 42 wherein said enzyme is selected from the group consisting of horseradish peroxidase and alkaline phosphatase.

48. The method of claim 43 wherein said enzyme substrate is selected from the group consisting of TMB (3,3′, 5,5′-tetramethyl benzidine), DAB (3,3′-Diaminobenzidine), ABTS (2,2′-Azino-bis(3-ethylbenzthiazoline-6-sulphonic acid)), AEC (3-Amino-9-ethylcarbazol), NBT (Nitro Blue Tetrazolium), CN/DAB (4-chloronaphthol/3,3′-diaminobenzidine, tetrahydrochloride), and 4-CN (4-chloro-1-napthol).

49. The method of claim 48 wherein said detectable signal is a precipitate, which adheres to the target cell.

50. The method of claim 39 wherein said target cell is selected from the group comprising white blood cells, tumor cells, bacteria, virus, unicellular organisms, cell from tissues and cells from organs.

51. A method of detecting and quantifying a target cell, said method comprising the steps of:
providing a bio-disc including a substantially circular substrate having a center and an outer edge, a target zone disposed between the center and the outer edge, an affinity agent bound to said substrate within said target zone such that said affinity agent is immobilized in said target zone;
providing a sample containing said target cell, said target cell having a surface marker;
mixing a reporter agent with said sample, said reporter agent having attached thereto a capture antibody and a binding agent, said capture antibody having affinity to said surface marker, said binding agent having affinity to said affinity agent;
allowing said capture antibody to bind with said surface marker on said target cell to thereby tag said target cell with said reporter agent,
depositing said sample with said tagged target cell on said target zone;
allowing said binding agent attached to said reporter agent to bind with said affinity agent to immobilize said target cell in said target zone;
washing said target zone to remove unbound cells; and
scanning a beam of electromagnetic radiation over said target zone to thereby determine the number of cells tagged with said reporter agent.

52. The method of claim 51 wherein said target cell is selected from the group comprising white blood cells, tumor cells, bacteria, virus, unicellular organisms, cell from tissues and cells from organs.

53. The method of claim 52 wherein said affinity agent is biotin.

54. The method of claim 52 wherein said binding agent is selected from the group consisting of streptavidin and neutravidin.
